(19) United States
(12) Patent Application Publication
(10) Pub. No.: US 2021/0071193 A1
(43) Pub. Date: Mar. 11, 2021

(54) PLANTS WITH ENHANCED RESISTANCE TO BACTERIAL PATHOGENS

(71) Applicant: The Regents of the University of California, Oakland, CA (US)

(72) Inventors: DOUGLAS DAHLBECK, CASTRO VALLEY, CA (US); ARTURO ORTEGA, BERKELEY, CA (US); ALEX SCHULTINK, BERKELEY, CA (US); BRIAN J. STASKAWICZ, BERKELEY, CA (US)

(21) Appl. No.: 16/925,649

(22) Filed: Jul. 10, 2020

Related U.S. Application Data
(60) Provisional application No. 62/873,582, filed on Jul. 12, 2019.

(51) Int. Cl. C12N 15/82; C12N 9/22

(52) U.S. Cl. CPC C12N 15/8281; C12N 9/22; C12N 15/8282

(57) ABSTRACT

The invention provides plants and seeds with enhanced resistance to plant disease caused by plant pathogens, particularly bacterial plant pathogens, and methods for producing such plants and seeds. The plants and seeds comprise mutated alleles of the Bs5 gene and/or Bs5-like gene that are capable of conferring to plants comprising the alleles enhanced resistance to plant pathogens. The present invention further provides methods of using the plants and seeds in agricultural production to limit plant diseases caused by plant pathogens.

### CROSS-REFERENCE TO RELATED APPLICATION

This application claims the benefit of U.S. Provisional Patent Application No. 62/873,582, filed Jul. 12, 2019, which is hereby incorporated herein in its entirety by reference.

### REFERENCE TO A SEQUENCE LISTING SUBMITTED AS A TEXT FILE

The official copy of the sequence listing is submitted electronically via EFS-Web as an ASCII formatted sequence listing with a file named 082313-0002SEQLST.TXT, created on Jul. 9, 2020 and having a size of 39.3 kilobytes, and is filed concurrently with the specification. The sequence listing contained in this ASCII-formatted document is part of the specification and is herein incorporated by reference in its entirety.

### FIELD OF THE INVENTION

The present invention relates to the field of plant improvement, specifically to the production of plants comprising enhanced resistance to plant diseases caused by plant pathogens, particularly bacterial plant pathogens.

### BACKGROUND OF THE INVENTION

Xanthomonas spp. are the causative pathogens of bacterial spot and have caused major yield losses in commercial varieties of tomato and pepper. Bacterial spot disease causes necrotic lesions on the leaf, stem, and fruits of the plant. Bacterial spot disease is a threat to worldwide production of Solanaceous crops, especially in warm and humid environments. Sustaining efficient productivity is essential for maintaining long-term food security. Most disease resistance genes that have been introgressed into crops for disease control have been genes that encode nucleotide binding leucine rich repeat proteins (NLRs) or pattern recognition receptors proteins (PRRs). Although dominant disease resistance genes have been deployed into commercial varieties of pepper, bacterial spot remains a threat as hypervirulent strains of the pathogen have evolved to evade dominant resistance genes.

Efforts have been made to research and deploy novel genes to provide durable resistance. The bacterial spot 5 (Bs5) gene is conserved among Solanaceae, is composed of three exons, and encodes a proline-rich putative transmembrane protein with an unknown function. Through bioinformatic analysis, the BS5 protein was predicted to belong to a superfamily of cysteine-rich transmembrane (CYSTM) proteins with diverse functions including stress and disease tolerance. A naturally occurring recessive allele (Cabs5) was found to confer resistance against a hypervirulent strain of Xanthomonas euvesicatoria in pepper (Capsicum annuum) (Jones et al. (2002) Phytopathology 92(3):273-277). The bs5 resistance phenotype is characterized by restricted pathogen growth and an absence of the hypersensitive response typically seen in NLRs. The hypersensitive response involves localized cell death at the site of infection. Pepper varieties containing the bs5 allele have shown durable resistance, impeding the emergence of bs5-resistant strains in commercial fields. The molecular basis for bs5 resistance at the protein level is still unknown.

The causative mutation of Cabs5 is an in-frame, six-nucleotide deletion in the third exon. This results in a double-leucine deletion at the C-terminus within the putative transmembrane domain (WO 2014/068346). CaBs5 is adjacent to a CaBs5 paralog (CaBs5-L), likely the result of gene duplication. CaBs5 and CaBs5-L encoded proteins have pairwise identity of 86 percent in which five amino acids are absent from 37-42 positions in the CaBs5-L encoded protein. The Cabs5 six-nucleotide deletion allele has only been found in the CaBs5 gene, suggesting no suppressive effects on the recessive Cabs5 resistance by the CaBs5-L gene. Orthologs of pepper CaBs5 and CaBs5-L have been previously identified in tomato (Solanum lycopersicum) (WO 2014/068346). Although the in-frame, six-nucleotide deletion that causes the double-leucine deletion is not known to occur naturally in tomato, it is have been reported that tomato plants displayed increased resistance to Xanthomonas euvesicatoria when the plants were transformed with the tomato bs5 (Slbs5) and the tomato bs5-L (Slbs5-L) transgenes each comprising the in-frame, six-nucleotide deletion and transgenes designed for the expression of artificial microRNAs (amiRNAs) designed to eliminate or reduce the expression of the native (wild-type) SLBS5 and SLBS5-L proteins from the genomic copies of the corresponding SlBs5 and SlBs5-L genes, respectively (WO 2014/068346). Thus, deployment of the bs5 allele in tomato has the potential to confer disease resistance against bacterial spot. Pepper and tomato are not sexually compatible, thus classical plant breeding strategies cannot be employed to establish bs5 resistance in tomato.

Targeted genome engineering has emerged as an alternative to classical breeding methods to improve crop plants and ensure sustainable food production. Targeted genome engineering based on the bacterial CRISPR (clustered regularly interspaced short palindromic repeats)/Cas (CRISPR-associated) type II prokaryotic adaptive immune system has become a popular tool for gene editing in plants (Liu and Fan (2014) Plant Mol. Biol. 85:209-218;). The first uses of CRISPR/Cas9 editing in plants were reported in 2013, with successful application for both transient expression and the recovery of stable, genome-edited lines (Li et al. (2013) Nat. Biotechnol. 31:688-691; Nekrasov et al. (2013) Nat. Biotechnol. 31:691-693). These technologies have provided the means to develop disease resistance beyond the limits imposed by classical breeding and without resorting to stably incorporated transgenes in the genomes of plants (Mushtaq et al. (2019) Front. Plant Sci. 10:550).

### BRIEF SUMMARY OF THE INVENTION

The present invention provides plants and seeds comprising enhanced resistance to plant diseases caused by plant pathogens, particularly bacterial plant pathogens. The plants and seeds of the present invention comprise at least one mutated allele of a Bs5 or a Bs5-like (Bs5-L) gene, wherein the mutated allele comprises a mutation in the portion of the Bs5 or Bs5-L gene corresponding to exon 2 or exon 3 of the tomato Bs5 (SlBs5) gene (SEQ ID NO: 1). Preferably, the at least one mutated allele is capable of conferring to the plant or seed enhanced resistance to at least one plant pathogen, relative to the resistance of a control plant lacking the at least one mutated allele. Preferred plants and seeds comprise in their genomes a Bs5 gene such as, for example, solanaceous plants, particularly solanaceous plants used in agriculture. In one embodiment, the present invention provides tomato plants comprising both a mutated bs5 allele and a mutated bs5-L allele and having enhanced resistance to one, two, three, or more bacterial plant pathogens when the tomato plants are homozygous for the mutated alleles.

The present invention further provides methods for producing a plant with enhanced resistance to at least one plant pathogen. The methods comprise inducing a mutation at a specific location within at least one of the Bs5 and Bs5-L genes in the genome of a plant cell, whereby at least one mutated allele of the Bs5 gene or the Bs5-L gene is produced. The methods further comprise regenerating a plant from the plant cell, wherein the plant comprises in its genome the at least one mutated allele. Preferably, the mutations within the Bs5 and Bs5-L genes are made by inducing single-strand or double-strand breaks at specific locations within these genes using a genome editing technology such as, for example, a CRISPR/Cas system, a transcription activator-like effector nuclease (TALEN), a zinc-finger nuclease (ZFN), or a homing endonuclease.

The present invention additionally provides methods of using the plants and seeds of the present invention in agriculture to limit plant diseases caused by plant pathogens. Such methods comprise planting a plant or seed of the present invention, for example, in a field or in a pot in a greenhouse and growing the plant or seed under conditions favorable for the growth and development of the plant. If desired, the methods can further comprise harvesting the part(s) of the plant that is/are typically used for human and/or animal consumption such as, for example, seeds, fruits, leaves, stems, tubers, roots, and flowers from the plant.

Additionally provided are isolated nucleic acid molecules, expression cassettes, and vectors, and host cells comprising nucleotide sequences of the mutated alleles of the present invention.

### DESCRIPTION OF THE DRAWINGS

FIG. 1 is a schematic showing the architecture of the Bs5/Bs5-L gene in both tomato and pepper and the Bs5/Bs5-L genomic targets. GT1 and GT2 refers to PAM sites targeted by guide RNA in the Cas9 vector constructs discussed in the Example 1 below.

FIG. 2 provides the amino acid sequence of the protein encoded by the wild-type tomato Bs5 (SlBs5) gene, and the amino acid sequences encoded by the bs5 alleles in tomato Lines 1-4 that were produced by targeted mutagenesis as discussed in the Examples below. In the amino acid sequences of the bs5 alleles in Lines 1-4, amino acids that are deleted, relative to the wild-type BS5 amino acid sequence, are depicted by hyphens, and amino acids that are not identical to the amino acid at the same position in the wild-type BS5 amino acid sequence, are depicted in bold. The type and exonic location of the modification in each of the lines are indicated in the columns labeled “Allele” and “Exon,” respectively.

FIG. 3 provides the amino acid sequence of the protein encoded by the wild-type tomato Bs5-like (SlBs5-L) gene, and the amino acid sequences encoded by the Slbs5 alleles in Lines 1-4 that were produced by targeted mutagenesis as discussed in the Examples below. In the amino acid sequences of the Slbs5 alleles in Lines 1-4, amino acids that are deleted, relative to the wild-type BS5-L amino acid sequence, are depicted by hyphens, and amino acids that are not identical to the amino acid at the same position in the wild-type BS5-L amino acid sequence, are depicted in bold. The type and exonic location of the modification in each of the lines are indicated in the columns labeled “Allele” and “Exon,” respectively.

FIG. 4 is a graphical representation of a Xanthomonas euvesicatoria growth assay on leaves of Line 1 vs. a control, susceptible variety (FL8000) at 14 days post inoculation (dpi). Line 1 shows a 10-fold decrease in colony forming units of X. euvesicatoria strain 85-10 per cm² leaf disc compared to susceptible wild-type tomato. The bacterial growth assay was initiated by dipping fully expanded tomato leaves that are attached to 3-4-week-old plants for 30 seconds in a bacterial solution having an OD600 of 0.2 and comprising 10 mM MgCl2 and 0.025% surfactant Silwet-L77. The plants were kept in a growth chamber for about 14-20 days or until disease symptoms were observed on leaves of a susceptible control, tomato plant. The growth chamber was maintained at 25° C., relative humidity of 50%, and 16 hour day/8 hour night photoperiod.

FIG. 5 is a graphical representation of a X. euvesicatoria growth assay on leaves of Line 2 vs. a control, susceptible variety (M82) at 14 dpi. Line 2 shows a 10-fold decrease in colony forming units of X. euvesicatoria strain 85-10 per cm² leaf disc compared to susceptible wild-type tomato. The growth assay was performed as described above the description of FIG. 4.

FIG. 6 is a photographic illustration of tomato leaves showing symptoms from the X. euvesicatoria growth assay of Line 2. Line 2 shows significantly less bacterial spot lesions compared to susceptible wild-type tomato (M82). A graphical representation of the results of the growth assay are provided in FIG. 4.

FIG. 7 is a graphical representation of a X. euvesicatoria growth assay on leaves of Line 3 vs. a control, susceptible variety (M82) at 14 dpi. Line 3 shows a 10-fold decrease in colony forming units of X. euvesicatoria strain 85-10 per cm² leaf disc compared to susceptible wild-type tomato.

FIG. 8 is a photographic illustration of an Xanthomonas perforans growth assay on Line 1 and a control susceptible plant (FL8000) at 14 dpi. Line 1 shows significantly less bacterial spot lesions compared to susceptible wild-type tomato (FL8000). The growth assay was performed essentially as described above the description of FIG. 4 but X. perforans was used instead of X. euvesicatoria.

FIG. 9 provides the results of a field trial conducted in Florida in which resistant tomato Line 1 was compared to a FL8000, a tomato line that is susceptible to bacterial spot disease caused by Xanthomonas euvesicatoria. The trial was conducted during the Fall 2018 growing season. The season began with typically rainy weather through September, and disease pressure was sufficient for observing differences among treatments. The weather was drier from early October onwards, and there was a corresponding reduction in bacterial spot disease severity. Average temperatures were unseasonably high from late September through October, resulting in low fruit set early in the season. Thus, total yields for the trial were low, as is reflected in the data. Nevertheless, Line 1 displayed a higher marketable yield fruit than susceptible line FL8000.

FIGS. 10A-10C are graphical representations that show the broad-spectrum resistance of Line 1 against other species of Xanthomonas. Bacterial growth assays were conducted using syringe infiltration instead of dip inoculation on fully expanded leaves in 4-week-old tomatoes. For Xanthomonas, leaves were macerated 8 days post infiltration. For Pseudomonas syringae race 2, leaves were harvested 6 days post infiltration. Line 1 showed resistance (10-fold reduction) against Xanthomonas gardneri (Xg153) (FIG. 10A) and Xanthomonas perforans (Xp4b) (FIG. 10B) but not against P. syringae (Pst R2) (FIG. 10C). A tomato line with the resistance gene BS2 (FL8000 BS2) was also included for comparison.

FIG. 11 is a bacterial growth time course. Fully expanded leaves from Line 1 were syringe infiltrated with X. gardneri. Leaf material was harvested over the course of 7 days to test the rate of bacterial growth. The susceptible wild-type tomato (solid line) shows a higher rate of bacterial growth over a week compared to Line 1 (dashed line), resulting in about a 10-fold reduction in bacterial populations in Line 1 at day 7. Bacterial spot symptoms usually begin a week after infiltration.

FIGS. 12A-12B are graphical representations of bacterial growth assays of Line 4 and heterozygous cross Line 4×WT. Fully expanded leaves were syringe infiltrated with X. euvesicatoria (Xe 85-10) (FIG. 12A) or X. perforans (Xp4b) (FIG. 12B) and harvested after 7 days. Line 4 (L4) showed a 10-fold reduction in internal bacterial populations compared to wildtype susceptible tomatoes and L4×FL8000 heterozygous cross. These results indicate that the mutations provide superior resistance when plants are homozygous for the mutant allele(s).

### SEQUENCE LISTING

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

SEQ ID NO: 1 sets forth the nucleotide sequence of the tomato (Solanum lycopersicum) Bs5 gene (SlBs5).

SEQ ID NO: 2 sets forth the nucleotide sequence of the coding region of SlBs5. If desired, a stop codon (e.g. TAA, TAG, or TGA) can be operably linked to the 3′ end of a nucleic acid molecule comprising SEQ ID NO: 2. It is noted that the native stop codon of SlBs5 is TGA.

SEQ ID NO: 3 sets forth the amino acid sequence of the SLBS5 protein encoded by SlBs5.

SEQ ID NO: 4 sets forth the nucleotide sequence of the tomato (Solanum lycopersicum) Bs5-like gene (SlBs5-L).

SEQ ID NO: 5 sets forth the nucleotide sequence of the coding region of SlBs5-L. If desired, a stop codon (e.g. TAA, TAG, or TGA) can be operably linked to the 3′ end of a nucleic acid molecule comprising SEQ ID NO: 5. It is noted that the native stop codon of SlBs5-L is TGA.

SEQ ID NO: 6 sets forth the amino acid sequence of the SLBS5-L protein encoded by SlBs5-L.

SEQ ID NO: 7 sets forth the nucleotide sequence of the allele of Slbs5 that is present in tomato Line 1.

SEQ ID NO: 8 sets forth the nucleotide sequence of the coding region of the allele of Slbs5 that is present in tomato Line 1. If desired, a stop codon (e.g. TAA, TAG, or TGA) can be operably linked to the 3′ end of a nucleic acid molecule comprising SEQ ID NO: 8. It is noted that the native stop codon of this allele is TAG.

SEQ ID NO: 9 sets forth the amino acid sequence of the protein encoded by allele of Slbs5 that is present in tomato Line 1.

SEQ ID NO: 10 sets forth the nucleotide sequence of the allele of Slbs5-L that is present in tomato Line 1.

SEQ ID NO: 11 sets forth the nucleotide sequence of the allele of Slbs5-L that is present in tomato Line 1. If desired, a stop codon (e.g. TAA, TAG, or TGA) can be operably linked to the 3′ end of a nucleic acid molecule comprising SEQ ID NO: 11. It is noted that the native stop codon of SlBs5-L is TAA.

SEQ ID NO: 12 sets forth the amino acid sequence of the protein encoded by the allele of Slbs5-L that is present in tomato Line 1.

SEQ ID NO: 13 sets forth the nucleotide sequence of the allele of Slbs5 that is present in tomato Line 2.

SEQ ID NO: 14 sets forth the nucleotide sequence of the coding region of the allele of Slbs5 that is present in tomato Line 2. If desired, a stop codon (e.g. TAA, TAG, or TGA) can be operably linked to the 3′ end of a nucleic acid molecule comprising SEQ ID NO: 14. It is noted that the native stop codon of this allele is TGA.

SEQ ID NO: 15 sets forth the amino acid sequence of the protein encoded by allele of Slbs5 that is present in tomato Line 2.

SEQ ID NO: 16 sets forth the nucleotide sequence of the allele of Slbs5-L that is present in tomato Line 2.

SEQ ID NO: 17 sets forth the nucleotide sequence of the allele of Slbs5-L that is present in tomato Line 2. If desired, a stop codon (e.g. TAA, TAG, or TGA) can be operably linked to the 3′ end of a nucleic acid molecule comprising SEQ ID NO: 17. It is noted that the native stop codon of SlBs5-L is TAA.

SEQ ID NO: 18 sets forth the amino acid sequence of the protein encoded by the allele of Slbs5-L that is present in tomato Line 2.

SEQ ID NO: 19 sets forth the nucleotide sequence of the allele of Slbs5 that is present in tomato Line 3.

SEQ ID NO: 20 sets forth the nucleotide sequence of the coding region of the allele of Slbs5 that is present in tomato Line 3. If desired, a stop codon (e.g. TAA, TAG, or TGA) can be operably linked to the 3′ end of a nucleic acid molecule comprising SEQ ID NO: 20. It is noted that the native stop codon of this allele is TGA.

SEQ ID NO: 21 sets forth the amino acid sequence of the protein encoded by allele of Slbs5 that is present in tomato Line 3.

SEQ ID NO: 22 sets forth the nucleotide sequence of the allele of Slbs5-L that is present in tomato Line 3.

SEQ ID NO: 23 sets forth the nucleotide sequence of the allele of Slbs5-L that is present in tomato Line 3. If desired, a stop codon (e.g. TAA, TAG, or TGA) can be operably linked to the 3′ end of a nucleic acid molecule comprising SEQ ID NO: 23. It is noted that the native stop codon of SlBs5-L is TAA.

SEQ ID NO: 24 sets forth the amino acid sequence of the protein encoded by the allele of Slbs5-L that is present in tomato Line 3.

SEQ ID NO: 25 sets forth the gRNA binding site in exon 2 of both SlBs5 and SlBs5-L that was used for CRISPR/cas9-mediated targeted mutagenesis as discussed in the Example 1 below. The protospacer-adjacent motif (PAM) consists of the last three nucleotides (i.e. GGG) at the 3′ end of SEQ ID NO: 25.

SEQ ID NO: 26 sets forth the gRNA binding site in exon 3 of both SlBs5 and SlBs5-L that was used for CRISPR/cas9-mediated targeted mutagenesis as discussed in Example 1 below. The protospacer-adjacent motif (PAM) consists of the last three nucleotides (i.e. TGG) at the 3′ end of SEQ ID NO: 26.

SEQ ID NO: 27 sets forth the nucleotide sequence of the allele of Slbs5 that is present in tomato Line 4.

SEQ ID NO: 28 sets forth the nucleotide sequence of the coding region of the allele of Slbs5 that is present in tomato Line 4. If desired, a stop codon (e.g. TAA, TAG, or TGA) can be operably linked to the 3′ end of a nucleic acid molecule comprising SEQ ID NO: 28. It is noted that the native stop codon of this allele is TGA.

SEQ ID NO: 29 sets forth the amino acid sequence of the protein encoded by allele of Slbs5 that is present in tomato Line 4.

SEQ ID NO: 30 sets forth the nucleotide sequence of the allele of Slbs5-L that is present in tomato Line 4.

SEQ ID NO: 31 sets forth the nucleotide sequence of the allele of Slbs5-L that is present in tomato Line 4. If desired, a stop codon (e.g. TAA, TAG, or TGA) can be operably linked to the 3′ end of a nucleic acid molecule comprising SEQ ID NO: 31. It is noted that the native stop codon of SlBs5-L is TAA.

SEQ ID NO: 32 sets forth the amino acid sequence of the protein encoded by the allele of Slbs5-L that is present in tomato Line 4.

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The present invention provides compositions and methods relating to plants and seeds comprising enhanced resistance to plant diseases caused by plant pathogens. The plants and seeds of the present invention find use in agriculture, particularly in limiting plant diseases caused by one or more plant pathogens. The plants and seeds of the present invention find further use in reducing or eliminating the need to apply synthetic, agricultural chemicals to crop plants to control plant diseases.

The present invention is based in part of the surprising discovery by the present inventors of new alleles of the tomato Bs5 and Bs5-L genes (SlBs5 and SlBs5-L, respectively) that are capable of conferring to tomato plants enhanced resistance to diseases caused by bacterial plant pathogens. Using a genome-editing approach to mutate wild-type alleles of the SlBs5 and SlBs5-L genes, the present inventors created three new, recessive resistance alleles that are unexpectedly different from the naturally occurring (Capsicum annuum) pepper bs5 (Cabs5) recessive resistance gene. Relative to the wild-type CaBs5 gene, the Cabs5 gene comprises the an in-frame, six-base-pair deletion in exon 3 that results in the loss of two consecutive leucine residues in CYSTM region at the C-terminal end of the protein encoded by the Cabs5 gene (WO 2014/068346). This deletion has been become known as the “double-Leu deletion.” As disclosed in further detail hereinbelow, none of the new mutated alleles of the SlBs5 and SlBs5-L genes comprise the double-Leu deletion. Also discussed in further detail below, the mutated alleles of the SlBs5 and SlBs5-L genes in tomato Line 1 comprise mutations that in exon 2 of the SlBs5 and SlBs5-L genes that alter the reading frame. These two mutated alleles encode proteins of 70 (Slbs5) and 94 (Slbs5-L) amino acids that comprise the first 31 amino acids of the amino acid sequences of the SLBS5 and SLBS5-L proteins, respectively, but share no homology to the amino acid sequences of the respective wild-type proteins after that point. Surprisingly, tomato plants of Line 1 displayed resistance to three different bacterial pathogens and displayed no detrimental phenotype as discussed in further detail in the Example 1 below.

In one aspect, the present invention provides plants or seeds with enhanced resistance to at least one plant pathogen. The plants or seeds comprise at least one mutated allele of a Bs5 or Bs5-L gene, wherein the mutated allele comprises a mutation in the portion of the Bs5 or Bs5-L gene corresponding to exon 2 or exon 3 of the SlBs5 gene (SEQ ID NO: 1). Such mutated alleles are capable of conferring to the plant enhanced resistance to at least one plant pathogen, relative to the resistance of a control plant lacking the at least one mutated allele.

It is recognized that the portion of a Bs5 or Bs5-L gene of interest corresponding to exon 2 or exon 3 of the SlBs5gene (SEQ ID NO: 1) the can be identified by methods known to those of skill in the art or disclosed elsewhere herein such as, for example, nucleotide amino acid sequence alignments. The amino acid sequence of the protein encoded by the SlBs5gene is set forth in SEQ ID NO: 3.

As used herein, the term “Bs5 gene” intended to encompass any known Bs5 gene such as, for example, the CaBs5 and SlBs5 genes from pepper and tomato, respectively, and orthologs of such known Bs5 genes that occur in any plant species. As used herein, “orthologs” are genes which are derived from a common ancestral gene, are found in different species as a result of speciation, and encode proteins with the same or similar function. As used herein, a “Bs5-L gene” is a paralog of particular Bs5 gene. While paralogs are homologous genes that are believed to have evolved by duplication of a gene, the present invention does not depend on a particular Bs5-L gene having been evolved by the duplication of a particular Bs5 gene. Moreover, the present invention does not depend on a plant even having a Bs5-L gene.

In preferred embodiments of the present invention, the mutated alleles of the present invention are recessive resistance alleles. Although most known resistance (R) genes are dominant R genes, recessive R genes may provide a more durable resistance because a loss of interaction of a key host susceptibility factor with a pathogen effector is a common mechanism for recessive R genes (Kourelis and van der Hoorn (2018) Plant Cell 30:285-299). While the mutated alleles of the present invention can be recessive resistance alleles (i.e. recessive R genes), the compositions and methods of the present invention do not depend on the resistance conferred on plants by the mutated alleles as having any particular biological mechanism such as, for example, loss of interaction of a key host susceptibility factor with a pathogen effector.

The mutated alleles of the present invention comprise mutations within exons 2 and 3 of the Bs5 and Bs5-L genes. The mutations include, for example, insertions of one or more nucleotides and/or deletions of one or more nucleotides, relative to the nucleotide sequence of the corresponding Bs5 or Bs5-L gene. Preferred mutations are those that result in changes in the amino acid sequences of BS5 and BS5-L, whereby the mutated bs5 or bs5-L allele encodes a protein that is not identical to the amino acid sequence of BS5 or BS5-L. Such non-identical amino acid sequences encoded by the mutated alleles have less than 100% amino acid sequence identity across the full-length of the respective amino acid sequence of BS5 or BS5-L. Such changes in the amino acid sequences encoded by the mutated alleles include, for example, additions of one or more amino acids, deletions of one or more amino acids, and/or substitutions of one or more amino acids.

Preferably, the mutated allele does not consist of a six-nucleotide deletion of the portion its nucleotide sequence (relative to the nucleotide sequence of the corresponding Bs5 or Bs5-L wild-type allele) corresponding to the two leucines at amino acid positions 88 and 89 in the tomato BS5 protein (SEQ ID NO: 3) or positions 86 and 87 in the tomato BS5-L protein (SEQ ID NO: 6).

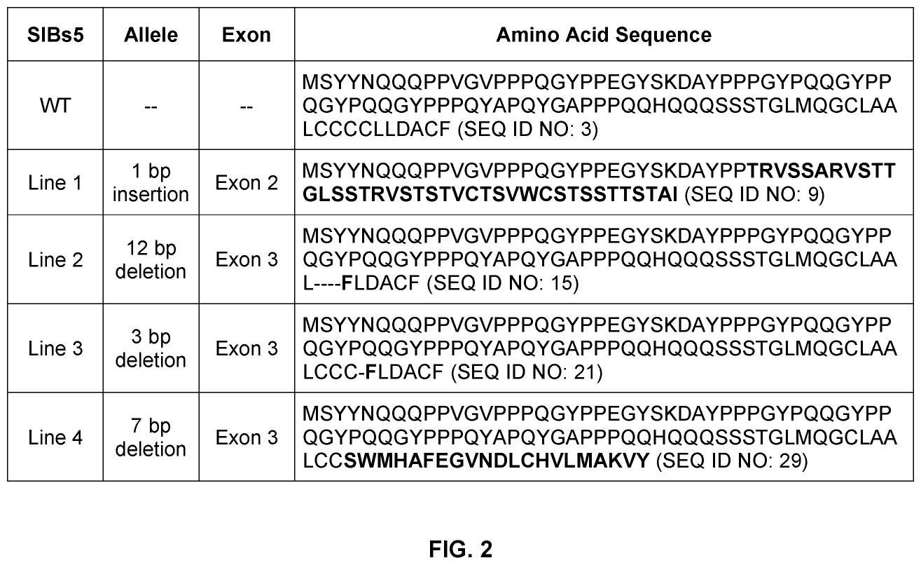

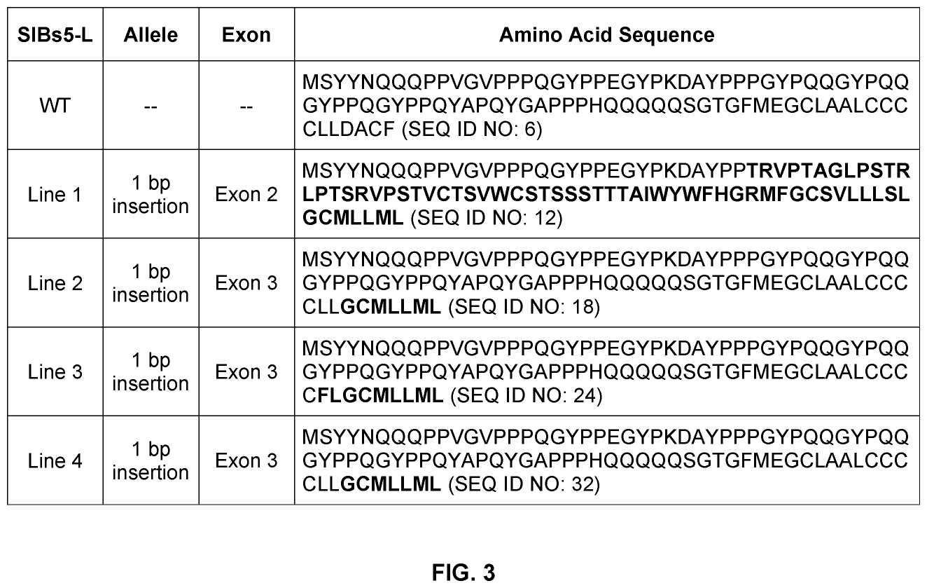

In some embodiments of the invention, the mutated allele encodes an amino acid sequence that is identical to the corresponding amino acid sequence of Bs5 or Bs5-L for about the first third of the amino acid sequence but shares little or no homology after that point. See FIGS. 2 and 3 and the amino acid sequences set forth in SEQ ID NOS: 9 and 12. In certain embodiments of the invention, the mutated allele encodes an amino acid sequence that is identical to the corresponding amino acid sequence of Bs5 or Bs5-L over the first (beginning from the N-terminus) 25, 26, 27, 28, 29, 30, 31, 32, 33, 34, 35, or 36 amino acids but shares little or no homology after that point in the amino acid sequence. See FIGS. 2 and 3 and the amino acid sequences set forth in SEQ ID NOS: 9 and 12. In certain other embodiments of the invention, the mutated allele encodes an amino acid sequence that is identical to the corresponding amino acid sequence of Bs5 or Bs5-L over the first 80, 81, 82, 83, 84, 85, 86, 87, or 88 amino acids but shares little or no homology after that point in the amino acid sequence. See FIGS. 2-3 and the amino acid sequences set forth in SEQ ID NOS: 18, 24, 29, and 32.

In one embodiment, the present invention provides tomato plants and seeds with enhanced resistance to one or more pathogens, particularly bacterial plant pathogens. Preferred bacterial plant pathogens of tomato include, but are not limited to, Xanthomonas spp. (e.g. X. euvesicatoria, X. perforans, X. gardneri) Pseudomonas spp. (e.g. P. syringae pv. tomato), and Ralstonia spp. (e.g. Ralstonia solanacearum). Such tomato plants and seeds of the present invention include, but are not limited to, tomato Lines 1, 2, 3, and 4 which are discussed in further detail in the Examples below. Additional examples of tomato plants and seeds of the present invention include tomato plants and seeds comprising in their genomes a mutated Slbs5 allele and/or a mutated Slbs5-L allele, wherein: the mutated Slbs5 allele comprises the nucleotide sequence set forth in SEQ ID NO: 7, 13, 19, or 27 and/or encoding an amino acid sequence set forth in SEQ ID NO: 9, 15, 21, or 29; and the mutated Slbs5-L allele comprises the nucleotide sequence set forth in SEQ ID NO: 10, 16, 22, or 30 and/or encoding an amino acid sequence set forth in SEQ ID NO: 12, 18, 24, or 32. In certain embodiment of the invention, the tomato plants and seeds comprise in their genomes: a mutated Slbs5 allele comprising the nucleotide sequence set forth in SEQ ID NO: 7 and/or a mutated Slbs5-L allele comprising the nucleotide sequence set forth in SEQ ID NO: 10; a mutated Slbs5 allele comprising the nucleotide sequence set forth in SEQ ID NO: 13 and/or a mutated Slbs5-L allele comprising the nucleotide sequence set forth in SEQ ID NO: 16; a mutated Slbs5 allele comprising the nucleotide sequence set forth in SEQ ID NO: 19 and/or a mutated Slbs5-L allele comprising the nucleotide sequence set forth in SEQ ID NO: 22, or a mutated Slbs5 allele comprising the nucleotide sequence set forth in SEQ ID NO: 27 and/or a mutated Slbs5-L allele comprising the nucleotide sequence set forth in SEQ ID NO: 30; wherein the plants and seeds are homozygous for one or both the mutated Slbs5 allele and the mutated Slbs5-L allele.

In certain other embodiments of the invention, the tomato plants and seeds comprise in their genomes: a mutated Slbs5 allele comprising the nucleotide sequence set forth in SEQ ID NO: 7 and a mutated Slbs5-L allele comprising the nucleotide sequence set forth in SEQ ID NO: 10; a mutated Slbs5 allele comprising the nucleotide sequence set forth in SEQ ID NO: 13 and a mutated Slbs5-L allele comprising the nucleotide sequence set forth in SEQ ID NO: 16; a mutated Slbs5 allele comprising the nucleotide sequence set forth in SEQ ID NO: 19 and a mutated Slbs5-L allele comprising the nucleotide sequence set forth in SEQ ID NO: 22, or a mutated Slbs5 allele comprising the nucleotide sequence set forth in SEQ ID NO: 27 and a mutated Slbs5-L allele comprising the nucleotide sequence set forth in SEQ ID NO: 30; wherein the plants and seeds are homozygous for both the mutated Slbs5 allele and the mutated Slbs5-L allele.

In another aspect, the present invention provides methods for producing a plant with enhanced resistance to at least one plant pathogen. The methods comprise inducing a mutation at a specific location within at least one of the Bs5 and Bs5-L genes in the genome of a plant cell, whereby at least one mutated allele of the Bs5 gene or the Bs5-L gene is produced. While any method or technology for inducing mutations a specific location within the genome of a plant cell that is known in art can be used in the present invention, preferred methods or technologies include those methods or technologies that cause a single-strand or a double-strand break in the DNA at a specific location of interest in the genome of a plant cell such as, for example, the genome-editing technologies using the CRISPR/Cas system, transcription activator-like effector nucleases (TALENs), zinc-finger nucleases (ZFNs), or homing endonuclease. In a preferred embodiment of the methods of the present invention, the CRISPR/Cas9 system is used to induce mutations the Bs5 gene and/or Bs5-L gene in the genome of a plant cell. In a more preferred embodiment of the methods of the present invention, the CRISPR/Cas9 system is used to induce mutations the Bs5 gene and/or Bs5-L gene in the genome of a plant cell together with a guide RNA designed to bind to or recognize a target nucleotide sequence within exon 2 or exon 3 of the Bs5 gene and/or Bs5-L gene. Examples of such target nucleotide sequences within exon 2 or exon 3 are set forth SEQ ID NOS: 25 and 26, respectively.

The methods for producing a plant with enhanced resistance to at least one plant pathogen can further comprise regenerating a plant from the plant cell, wherein the plant comprises in its genome the at least one mutated allele. Preferably, the regenerated plant is homozygous for the at least one mutated allele. If desired, the regenerated plant can be selfed to produce progeny plants that are homozygous for the at least one mutated allele. The methods can further involve assaying the regenerated plants or progeny plants for enhanced resistance to at least one plant pathogen of interest using methods disclosed elsewhere herein or otherwise known in the art.

Preferably, plants comprising at least one mutated allele of the present invention comprise no significant deleterious effects on plant growth, development, and reproduction, disease resistance, insect resistance, environmental stress tolerance, agronomic yield, and the like.

The present invention is drawn to compositions and methods for enhancing the resistance of a plant to plant disease, particularly to compositions and methods for enhancing the resistance of a wheat plant to wheat stem rust. By “disease resistance” is intended that the plants avoid the disease symptoms that are the outcome of plant-pathogen interactions. That is, pathogens are prevented from causing plant diseases and the associated disease symptoms, or alternatively, the disease symptoms caused by the pathogen are minimized or lessened.

Definitions of some terms used herein include, but are not limited to, the following definitions:

“Backcrossing” is a process in which a breeder repeatedly crosses hybrid progeny, for example a first generation hybrid (F1), back to one of the parents of the hybrid progeny. Backcrossing can be used to introduce one or more single locus conversions from one genetic background into another.

“Crossing” is the mating of two parent plants. For the present invention, references to the crossing of a first plant to a second plant are not intended to imply that the first plant is the male parent (pollen donor) and the second plant is the female parent (egg donor) or that the first plant is the female parent and the second plant is the male parent unless expressly stated or apparent from the context of usage.

“Cross-pollination” is the fertilization by the union of two gametes from different plants.

A “diploid” is a cell or organism having two sets of chromosomes.

An “exogenous DNA molecule” is a single-stranded or double-stranded DNA molecule that does not occur naturally in a cell. The exogenous DNA molecule can be a linear DNA molecule or a circular DNA molecule. Typically, such exogenous DNA is introduced into the cell or into a progenitor of the cell.

An “F1 hybrid” is the first generation progeny of the cross of two nonisogenic plants.

A “genotype” is the genetic constitution of a cell or organism.

A “haploid” is a cell or organism having one set of the two sets of chromosomes in a diploid.

A “line” is a group of plants that are genetically identical or substantially the same genetically and is distinguished from any other plant grouping by the expression of at least one of characteristic or trait. For example, tomato Line 1 of the present invention that distinguished from FL8000 by specific mutations in the SlBs5 and SlBs5-L genes and by displaying enhanced resistance to certain bacterial plant pathogens.

A “mutated allele” of the Bs5 gene or Bs5-L gene comprises at least one addition, deletion, and/or substitution relative the nucleotide sequence of the corresponding Bs5 gene or Bs5-L gene. The mutated alleles of the present invention can be produced by the methods disclosed elsewhere herein. Preferably, the mutated alleles of the present invention are not known to occur in nature (i.e. non-naturally occurring) and do not include the in-frame, six-base-pair deletion in exon 3 that results in the loss of two consecutive leucine residues in CYSTM region at the C-terminal end of the encoded protein.

A “phenotype” is the detectable characteristics of a cell or organism, which characteristics are the manifestation of gene expression.

As used herein, the terms “resistance” and “tolerance” are used interchangeably to describe plants of the present invention that show no or lesser symptoms to a plant disease caused by a plant pathogen, particularly a bacterial fungal, oomycete, or fungal plant pathogen, when compared to a control plant lacking a mutated allele gene of the present invention. These terms are also used to describe plants showing some symptoms but that are still able to produce marketable product with an acceptable yield.

“Regeneration” is the development of a plant from a tissue of cell culture.

“Selfing” is the mating of a single plant with itself by self-pollination.

“Self-pollination” is the transfer of pollen from the anther to the stigma of the same plant.

A “transgene” is a genetic locus comprising a sequence which has been introduced into the genome of a plant by transformation.

Additional terms concerning the present invention are defined elsewhere herein above and below.

Genome-editing technologies that can be used to produce the plants and seeds of the present invention include, but are not limited to, methods involving targeted mutagenesis and homologous recombination. Targeted mutagenesis or similar techniques are disclosed in U.S. Pat. Nos. 5,565,350; 5,731,181; 5,756,325; 5,760,012; 5,795,972 and 5,871,984; all of which are herein incorporated in their entirety by reference. Methods for targeted mutagenesis and for gene modification or gene replacement involving homologous recombination can involve inducing single-strand or double-strand breaks in DNA using, for example, TAL (transcription activator-like) effector nucleases (TALEN), Clustered Regularly Interspaced Short Palindromic Repeats/CRISPR-associated nuclease (CRISPR/Cas nuclease), zinc-finger nucleases (ZFN), or homing endonucleases that have been engineered to make double-strand breaks at specific recognition sequences in the genome of a plant, other organism, or host cell. See, for example, Durai et al., (2005) Nucleic Acids Res 33:5978-90; Mani et al. (2005) Biochem Biophys Res Comm 335:447-57; U.S. Pat. Nos. 7,163,824, 7,001,768, and 6,453,242; Arnould et al. (2006) J Mol Biol 355:443-58; Ashworth et al., (2006) Nature 441:656-9; Doyon et al. (2006) J Am Chem Soc 128:2477-84; Rosen et al., (2006) Nucleic Acids Res 34:4791-800; and Smith et al., (2006) Nucleic Acids Res 34:e149; U.S. Pat. App. Pub. No. 2009/0133152; and U.S. Pat. App. Pub. No. 2007/0117128; all of which are herein incorporated in their entirety by reference.

It is recognized that a mutated allele of the present invention that has any particular nucleotide sequence can be produced in a plant of interest through the use of a genome-editing technology though a gene replacement approach. The mutated allele can be produced by introducing into a plant cell an exogenous DNA molecule comprising the mutation site of mutated allele and a suitable amount of nucleotides on either side of the mutation site, whereby following the induction of a double stranded break, homologous recombination occurs between the exogenous DNA and the wild-type allele of the Bs5 gene or Bs5-L is converted to the mutated allele. It is further recognized that such an approach to make be used to make the tomato plants comprising any of the mutated alleles of the Slbs5 and SlBs5-L genes having the nucleotide sequences set forth in SEQ ID NOS: 7, 10, 13, 16, 19, 22, 27, and 30.

The CRISPR/Cas nuclease system can also be used to make single-strand or double-strand breaks at specific recognition sequences in the genome of a plant for both targeted mutagenesis and for gene modification or gene replacement through homologous recombination. The CRISPR/Cas nuclease is an RNA-guided (simple guide RNA, sgRNA in short) DNA endonuclease system performing sequence-specific single-strand or double-strand breaks in a DNA segment homologous to the designed RNA. It is possible to design the specificity of the sequence (Cho S. W. et al., Nat. Biotechnol. 31:230-232, 2013; Cong L. et al., Science 339:819-823, 2013; Mali P. et al., Science 339:823-826, 2013; Feng Z. et al., Cell Research: 1-4, 2013).

TAL effector nucleases (TALENs) can be used to make double-strand breaks at specific recognition sequences in the genome of a plant for gene modification or gene replacement through homologous recombination. TAL effector nucleases are a class of sequence-specific nucleases that can be used to make double-strand breaks at specific target sequences in the genome of a plant or other organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. (2010) doi:10.1093/nar/gkq704; and Miller et al. (2011) Nature Biotechnology 29:143-148; all of which are herein incorporated by reference.

In addition, a ZFN can be used to make double-strand breaks at specific recognition sequences in the genome of a plant for targeted mutagenesis or for gene modification or gene replacement through homologous recombination. The Zinc Finger Nuclease (ZFN) is a fusion protein comprising the part of the FokI restriction endonuclease protein responsible for DNA cleavage and a zinc finger protein which recognizes specific, designed genomic sequences and cleaves the double-stranded DNA at those sequences, thereby producing free DNA ends (Urnov F. D. et al., Nat Rev Genet. 11:636-46, 2010; Carroll D., Genetics. 188:773-82, 2011).

Breaking DNA using site-specific nucleases, such as, for example, those described herein above, can increase the rate of homologous recombination in the region of the breakage. Thus, coupling of such effectors as described above with nucleases enables the generation of targeted changes in genomes which include additions, deletions and other modifications.

The present invention additionally provides methods for identifying a plant comprising a mutated bs5 or bs5-L allele of the present invention. The methods find use in breeding crop plants for resistance to plant pathogens, particularly bacterial plant pathogens. Such resistant plants find use in agricultural production. The methods for identifying a plant comprising a mutated bs5 or bs5-L allele of the present invention comprise detecting in a plant or in at one cell thereof the presence of at least one mutated bs5 or bs5-L allele of the present invention. In preferred embodiments, detecting the presence of a mutated bs5 or bs5-L allele comprises detecting the presence at least the mutation site in exon 2 or exon 3 of the mutated bs5 or bs5-L allele.

In the methods for identifying a plant comprising a mutated bs5 or bs5-L allele of the present invention, detecting the presence of the mutated bs5 or bs5-L allele in a plant can involve one or more of the following molecular biology techniques that are disclosed elsewhere herein or otherwise known in the art including, but not limited to, isolating genomic DNA and/or RNA from the plant, amplifying nucleic acid molecules comprising at least a portion of the mutated bs5 or bs5-L allele by PCR amplification, sequencing nucleic acid molecules comprising the mutated bs5 or bs5-L allele, identifying the mutated bs5 or bs5-L allele, or a transcript of the mutated bs5 or bs5-L allele by nucleic acid hybridization, and conducting an immunological assay for the detection of the protein encoded by the mutated bs5 or bs5-L allele. It is recognized that oligonucleotide probes and PCR primers can be designed to identity the mutated bs5 or bs5-L alleles of the present invention and that such probes and PCR primers can be utilized in methods disclosed elsewhere herein or otherwise known in the art to rapidly identify in a population of plants one or more plants comprising the presence of a mutated bs5 or bs5-L allele of the present invention.

In a preferred embodiment of the invention, the methods for identifying a plant comprising a mutated bs5 or bs5-L allele comprise detecting in a tomato plant the presence of a mutated bs5 allele having a nucleotide sequence selected from the group consisting for SEQ ID NOS: 7, 13, 19, and 27, a mutated bs5-L allele having a nucleotide sequence selected from the group consisting for SEQ ID NOS: 10, 16, 22, and 30, or both of said mutated alleles. Such methods find use in breeding new tomato lines with enhanced resistance to plant disease caused by plant pathogens, particularly bacterial plant pathogens.

One aspect of the current invention concerns methods for producing new plant lines and hybrids with enhanced resistance to at least one plant pathogen and comprising at least one mutated allele of the present invention. In some embodiments, the methods involve crossing a first plant comprising a mutated allele gene to a second plant lacking a mutated allele of a Bs5 and/or Bs5-L gene. The first plant can be homozygous or heterozygous for a mutated allele of a Bs5 or Bs5-L gene. However, in preferred embodiments the first plant is homozygous for a mutated allele of a Bs5 or Bs5-L gene whereby all F1 progeny of the crossing will comprise the mutated allele. If the first plant is heterozygous for a mutated allele of a Bs5 or Bs5-L gene, then the method can comprise the optional step of selecting for the presence of the mutated allele using, for example, PCR amplification of a genomic DNA amplicon comprising at least a portion of the mutated allele comprising the mutation site to identify one or more plants comprised the desired mutated allele. If desired, the methods can further involve selfing the F1 progeny plant so as to produce F2 progeny plants and then selecting at least one F2 progeny plant that is homozygous for the mutated allele of the Bs5 or Bs5-L gene using, for example, the PCR amplification or sequencing as described above or functional assays for disease resistance or enzyme activity. It is recognized the desired homozygous F2 progeny plants will display enhanced resistance to the one or more plant diseases.

In other embodiments of the invention, the present invention provides methods for producing hybrid seeds and plants, particularly hybrid seeds and plants that are homozygous for a mutated allele of the Bs5 and/or Bs5-L gene. Such methods involve crossing a first plant that is homozygous for a mutated allele of the present invention to a second plant that is homozygous for the same or a different mutated allele of the present invention whereby an F1 hybrid seed is produced that is homozygous for a mutated allele. Typically, the first and second plants are from different inbred lines that are not genetically identical, whereby the F1 progeny resulting from the crossing display hybrid vigor. For the present invention, a diploid plant or seed is considered homozygous for a mutated allele of the present invention if the plant or seed possesses a mutated allele at each of the two Bs5 or Bs5-L loci in the diploid genome, whether or not the two mutated alleles share the same nucleotide sequence. If desired, the methods can further involve producing a hybrid plant that is homozygous for mutated allele gene by planting the seed in soil or other growth medium under conditions favorable for the germination of the seed and the development of a seedling plant therefrom, whereby a hybrid plant is produced.

The development of new varieties using one or more starting lines is well known in the art. In accordance with the invention, novel plant varieties may be created by crossing a plant comprising a mutated allele of the present invention with a second plant followed by multiple generations of breeding according to methods well known in the art and/or described elsewhere herein. New varieties may be created by crossing with any second plant. In selecting such a second plant to cross for the purpose of developing novel lines, it may be desired to choose those plants which either themselves exhibit one or more selected desirable characteristics or which exhibit the desired characteristic(s) when in hybrid combination. Once initial crosses have been made, inbreeding and selection take place to produce new varieties. For development of a uniform line, often five or more generations of selfing and selection are involved.

Uniform lines of new varieties may also be developed by way of double-haploids. This technique allows the creation of true breeding lines without the need for multiple generations of selfing and selection. In this manner true breeding lines can be produced in as little as one generation. Haploid embryos may be produced from microspores, pollen, anther cultures, or ovary cultures. The haploid embryos may then be doubled autonomously, or by chemical treatments (e.g. colchicine treatment). Alternatively, haploid embryos may be grown into haploid plants and treated to induce chromosome doubling. In either case, fertile homozygous plants are obtained. In accordance with the invention, any of such techniques may be used in connection with a plant of the invention and progeny thereof to achieve a homozygous line.

Backcrossing can also be used to improve an inbred plant. Backcrossing transfers a specific desirable trait from one inbred or non-inbred source to an inbred that lacks that trait. This can be accomplished, for example, by first crossing a superior inbred (A) (recurrent parent) to a donor inbred (non-recurrent parent), which carries the appropriate locus or loci for the trait in question. The progeny of this cross are then mated back to the superior recurrent parent (A) followed by selection in the resultant progeny for the desired trait to be transferred from the non-recurrent parent. After five or more backcross generations with selection for the desired trait, the progeny have the characteristic being transferred, but are like the superior parent for most or almost all other loci. The last backcross generation would be selfed to give pure breeding progeny for the trait being transferred.

The tomato plants of the present invention are particularly well suited for the development of new tomato lines with enhanced resistance to plant disease caused by plant pathogens, particularly bacterial plant pathogens. In selecting a second plant to cross with a first plant comprising a mutated allele of the present invention for the purpose of developing novel tomato lines, it will typically be preferred to choose those plants which either themselves exhibit one or more selected desirable characteristics or which exhibit the desired characteristic(s) when in hybrid combination. Examples of desirable traits may include, in specific embodiments, high seed yield, high seed germination, seedling vigor, high fruit yield, disease tolerance or resistance, and adaptability for soil and climate conditions. Consumer-driven traits, such as a fruit shape, color, texture, and taste are other examples of traits that may be incorporated into new lines of tomato plants developed by this invention.

The tomato plants and seeds can comprise one or more other disease tolerance or resistance genes in addition to a mutated allele of the present invention. Any such disease tolerance or resistance genes that are known in the art and that are suitable for use in tomato plants can be used. Preferred disease tolerance or resistance genes include, for example, the Bs2, EFR, ZAR1, JIM2, and Roq1 resistance genes. See U.S. Pat. Nos. 6,262,343, 6,762,285, 9,222,103, and 9,816,103; Schultink et al. (2017) Plant J. 92(5):787-795; Schultink et al. (2019). New Phytol. 221(2):1001-1009; all of which are herein incorporated by reference.

Tomato plants and seeds of the present invention comprising a mutated allele of the SLBs5 gene and/or a mutated allele of the SlBs5-L gene, particularly tomato plants and seeds comprising the mutated allele(s) of the present invention in a homozygous condition, find particular use in commercial tomato production in areas in which plant diseases are prevalent and can provide increased yields of tomato fruit compared to tomato fruit yields with tomato plants lacking the mutated allele(s). Thus, the present invention further provides methods for increasing tomato fruit production in areas where such plant diseases are known to occur. The method comprises growing one or more tomato plants comprising a mutated allele of the SLBs5 gene and/or a mutated allele of the SlBs5-L gene under conditions favorable for the development of the plant disease. Typically, the tomato plants are grown outdoors in agricultural fields but the methods of the invention can also be used with tomato plants grown under greenhouse conditions or under other indoor or controlled-environment conditions. The tomato plants are allowed to grow and produce mature tomato fruit and then the fruit are harvested from the plants. The methods provide for an increase in tomato fruit yield in the presence of one plant diseases when compared to the tomato fruit yield of control tomato plants grown under the same or similar conditions.

The tomato plants comprising a mutated allele of the present invention, particularly tomato plants comprising a mutated allele in a homozygous condition, comprise enhanced resistance to at least one plant pathogen relative to the resistance control tomato plants lacking a mutated allele of the present invention.

The methods of the present invention find use in producing plants, particularly tomato plants, with enhanced resistance to a plant disease caused by at least one plant pathogen. Typically, the methods of the present invention will enhance or increase the resistance of the subject plant to one strain of a plant pathogen or to each of two or more strains of the plant pathogen by at least 25%, 50%, 75%, 100%, 150%, 200%, 250%, 500% or more when compared to the resistance of a control to same strain or strains of the plant pathogen. Unless stated otherwise or apparent from the context of a use, a control plant for the present invention does not comprise a mutated allele of present invention. Preferably, the control plant is essentially identical (e.g. same species, subspecies, and variety) to a plant comprising the mutated bs5 allele and/or the bs5-L allele of the present invention except the control plant does not comprise a mutated allele of present invention. More preferably, the control plant is essentially identical to a plant comprising a mutated bs5 allele and/or a bs5-L allele of the present invention, except that the control plant does not comprise a mutated allele of present invention and is homozygous for Bs5 and/or Bs5-L.

While the mutated bs5 and bs5-L alleles of the present invention are referred to herein as recessive resistance alleles and are capable of conferring to a plant enhanced resistance to one or more plant pathogens when the mutated bs5 allele and/or the mutated bs5-L allele is in the homozygous recessive state, the present invention does not depend on such mutated alleles conferring resistance to a plant only when in the heterozygous state. It is recognized that a plant which is heterozygous for a mutated bs5 allele and/or a mutated bs5-L allele of present invention may display a level of resistance to one or more plant pathogens that is higher than the level of resistance of a control plant that is homozygous for the (wild-type) Bs5 allele and/or the (wild-type) Bs5-L allele but lower (typically much lower) than the level of resistance displayed by a plant that is homozygous for the same mutated bs5 allele and/or the same mutated bs5-L allele. Typically, the resistance of the subject plant that homozygous for the mutated bs5 allele and/or the mutated bs5-L allele will display resistance that is enhanced or increased by at least 50%, 75%, 100%, 150%, 200%, 250%, 500% or more when compared to the resistance of a plant that is heterozygous for the same mutated allele(s) as the subject plant.

Unless expressly stated or apparent from the context of usage, the methods and compositions of the present invention can be used with any plant species including, for example, monocotyledonous plants, dicotyledonous plants, and conifers. Examples of plant species of interest include, but are not limited to, corn (Zea mays), Brassica sp. (e.g., B. napus, B. raga, B. juncea), particularly those Brassica species useful as sources of seed oil, alfalfa (Medicago sativa), rice (Oryza sativa), rye (Secale cereale), triticale (×Triticosecale or Triticum×Secale) sorghum (Sorghum bicolor, Sorghum vulgare), teff (Eragrostis tef), millet (e.g., pearl millet (Pennisetum glaucum), proso millet (Panicum miliaceum), foxtail millet (Setaria italica), finger millet (Eleusine coracana)), switchgrass (Panicum virgatum), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), strawberry (e.g. Fragaria×ananassa, Fragaria vesca, Fragaria moschata, Fragaria virginiana, Fragaria chiloensis), sweet potato (Ipomoea batatus), yam (Dioscorea spp., D. rotundata, D. cayenensis, D. alata, D. polystachya, D. bulbifera, D. esculenta, D. dumetorum, D. trifida), cassava (Manihot esculenta), coffee (Coffea spp.), coconut (Cocos nucifera), oil palm (e.g. Elaeis guineensis, Elaeis oleifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), date (Phoenix dactylifera), cultivated forms of Beta vulgaris (sugar beets, garden beets, chard or spinach beet, mangelwurzel or fodder beet), sugarcane (Saccharum spp.), oat (Avena sativa), barley (Hordeum vulgare), cannabis (Cannabis sativa, C. indica, C. ruderalis), poplar (Populus spp.), eucalyptus (Eucalyptus spp.), Arabidopsis thaliana, Arabidopsis rhizogenes, Nicotiana benthamiana, Brachypodium distachyon vegetables, ornamentals, and conifers and other trees. In specific embodiments, plants of the present invention are crop plants (e.g. maize, sorghum, wheat, millet, rice, barley, oats, sugarcane, alfalfa, soybean, peanut, sunflower, cotton, safflower, Brassica spp., lettuce, strawberry, apple, citrus, etc.).

Vegetables include tomatoes (Lycopersicon esculentum), eggplant (also known as “aubergine” or “brinjal”) (Solanum melongena), pepper (Capsicum annuum), lettuce (e.g., Lactuca sativa), green beans (Phaseolus vulgaris), lima beans (Phaseolus limensis), peas (Lathyrus spp.), chickpeas (Cicer arietinum), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Fruit trees and related plants include, for example, apples, pears, peaches, plums, oranges, grapefruits, limes, pomelos, palms, and bananas. Nut trees and related plants include, for example, almonds, cashews, walnuts, pistachios, macadamia nuts, filberts, hazelnuts, and pecans.

In specific embodiments, the plants of the present invention are crop plants such as, for example, maize (corn), soybean, wheat, rice, cotton, alfalfa, sunflower, canola (Brassica spp., particularly Brassica napus, Brassica rapa, Brassica juncea), rapeseed (Brassica napus), sorghum, millet, barley, triticale, safflower, peanut, sugarcane, tobacco, potato, tomato, and pepper.

In certain embodiments, the plants of the present invention are solanaceous plants. Solanaceous plants of the present invention include, but are not limited to, potato (Solanum tuberosum), eggplant (Solanum melongena), petunia (Petunia spp., e.g., Petunia×hybrida or Petunia hybrida), tomatillo (Physalis philadelphica), Cape gooseberry (Physalis peruviana), Physalis sp., woody nightshade (Solanum dulcamara), garden huckleberry (Solanum scabrum), gboma eggplant (Solanum macrocarpon), pepper (Capsicum spp.; e.g., Capsicum annuum, C. baccatum, C. chinense, C. frutescens, C. pubescens, and the like), tomato (Solanum lycopersicum or Lycopersicon esculentum), tobacco (Nicotiana spp., e.g. N. tabacum, N. benthamiana), Solanum americanum, Solanum demissum, Solanum stoloniferum, Solanum papita, Solanum bulbocastanum, Solanum edinense, Solanum schenckii, Solanum hjertingii, Solanum venturi, Solanum mochiquense, Solanum chacoense, and Solanum pimpinellifolium. In preferred embodiments of the methods and compositions of the present invention, the solanaceous plants are solanaceous plants grown in agriculture including, but not limited to, tomato, pepper, potato, tomatillo, eggplant, tobacco, Cape gooseberry, and petunia. In more preferred embodiments, the solanaceous plants are tomato and pepper. In even more preferred embodiments, the preferred plant is tomato.

The term “plant” is intended to encompass plants at any stage of maturity or development, as well as any cells, tissues or organs (plant parts) taken or derived from any such plant unless otherwise clearly indicated by context. Plant parts include, but are not limited to, fruits, stems, tubers, roots, flowers, ovules, stamens, petals, leaves, hypocotyls, epicotyls, cotyledons, embryos, meristematic regions, callus tissue, anther cultures, gametophytes, sporophytes, pollen, microspores, protoplasts, seeds, and the like. It is recognized that the plant protoplasts of the present invention can be prepared from any one or more of the aforementioned plant parts and at any stage of development and/or maturity.

Likewise, the term “plant cell” is intended to encompass plant cells obtained from or in plants at any stage of maturity or development unless otherwise clearly indicated by context. Plant cells can be from or in plant parts including, but are not limited to, fruits, stems, tubers, roots, flowers, ovules, stamens, leaves, embryos, meristematic regions, callus tissue, anther cultures, gametophytes, sporophytes, pollen, microspores, in vitro-cultured tissues, organs or cells and the like. It is recognized that the plant protoplasts of the present invention can be prepared from any one or more of the aforementioned plant cells and at any stage of development and/or maturity. As used herein, unless expressly stated otherwise or apparent from the context of usage, the term “plant cell” is intended to encompass a plant protoplast.

Pathogens of the present invention include, but are not limited to, bacterial, oomycete, fungal, and viral pathogens. Preferred pathogens are bacterial pathogens including, but not limited to, Xanthomonas gardneri, Xanthomonas perforans, Pseudomonas syringae pv. tomato, Ralstonia solanacearum, Xanthomonas euvesicatoria, Clavibacter michiganensis subsp. michiganensis, Xanthomonas campestris pv. vesicatoria, Envinia carotovora subsp. carotovora, Ralstonia solanacearum, Pseudomonas corrugata, Pseudomonas cichorii, and Pseudomonas syringae pv. syringae

Oomycete pathogens of the present invention include, but are not limited to, Phytophthora capsici, Phytophthora infestans, Phytophthora parasitica, Phytophthora ramorum, Phytophthora ipomoeae, Phytophthora mirabilis, Phytophthora capsici, Phytophthora porri, Phytophthora phaseoli, Phytophthora spp., Hyaloperonospora arabidopsidis, Hyaloperonospora parasitica, Bremia lactucae, Peronospora farinosa, Pseudoperonospora cubensis, Pseudoperonospora humuli, Peronospora destructor, Albugo candida, Albugo occidentalis, Pythium aphanidermatum, Pythium arrhenomanes, Pythium debaryanum, Pythium myriotylum, Pythium ultimum, and Pythium spp.

Fungal pathogens of the present invention include, but are not limited to, Colletotrichum coccodes, Colletotrichum dematium, Colletotrichum gloeosporioides, Colletotrichum spp., Cladosporium fulvum, Fusarium oxysporum f. sp. lycopersici, Fusarium oxysporum f. sp. radicis-lycopersici, Leveillula taurica/Oidiopsis taurica, Glomerella cingulata, Oidiopsis sicula, Verticillium albo-atrum Verticillium dahliae, Botrytis cinerea, Alternaria alternata, Stemphylium botryosum, Pleospora tarda, Stemphylium herbarum, Stemphylium botryosum f. sp. lycopersici, Stemphylium lycopersici, Stemphylium solani, Stemphylium spp., Pleospora herbarum, Ulocladium consortiale, Thielaviopsis basicola, Chalara elegans, Pseudocercospora fuligena, Phoma destructive, Rhizoctonia solani, Rhizoctonia spp., Athelia rolfsii, Corynespora cassiicola, Septoria lycopersici, Sclerotinia sclerotiorum, Sclerotinia minor, and Sclerotinia spp.

Viral pathogens of the present invention include, but are not limited to, tomato mosaic virus (ToMV), tobacco mosaic virus (TMV), curtovirus, potato virus Y, tomato pseudo-curly top virus, tomato bushy stunt virus, tobacco etch virus, cucumber mosaic virus, tomato mottle geminivirus, alfalfa mosaic virus, tomato spotted wilt virus, tomato yellow leaf curl virus, tomato yellow top virus, tomato bunchy top viroid, and tomato planto macho viroid.

The present invention encompasses isolated or substantially purified polynucleotide (also referred to herein as “nucleic acid molecule”, “nucleic acid” and the like) or protein (also referred to herein as “polypeptide”) compositions including, for example, polynucleotides and proteins comprising the sequences set forth in the accompanying Sequence Listing as well as variants and fragments of such polynucleotides and proteins. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Fragments and variants of the disclosed polynucleotides and proteins encoded thereby are also encompassed by the present invention. By “fragment” is intended a portion of the polynucleotide or a portion of the amino acid sequence and hence protein encoded thereby. Fragments of polynucleotides comprising coding sequences may encode protein fragments that retain biological activity of the full-length or native protein. Alternatively, fragments of a polynucleotide that are useful as hybridization probes generally do not encode proteins that retain biological activity or do not retain promoter activity. Thus, fragments of a nucleotide sequence may range from at least about 20 nucleotides, about 50 nucleotides, about 100 nucleotides, and up to the full-length polynucleotide of the invention.

Polynucleotides that are fragments of the mutated alleles of the present invention and their coding sequences comprise at least 16, 20, 50, 75, 100, 125, 150, 175, 200, 300, 400, 600, 800, 1000, 1200, 1400, 1600, 1800, or 1900 contiguous nucleotides, or up to the number of nucleotides present in a full-length mutated alleles of the Bs5 or Bs5-L genes or their coding sequence disclosed herein.

“Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the bs5 or bs5-L proteins of the invention. Naturally occurring allelic variants such as these can be identified with the use of well-known molecular biology techniques, as, for example, with polymerase chain reaction (PCR) and hybridization techniques as outlined below. Generally, variants of a particular polynucleotide of the invention will have at least about 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. In certain embodiments of the invention, variants of a particular polynucleotide of the invention will have at least about 80%, 85%, 90%, 91%, 92%, 93%, 94%, 95%, 96%, 97%, 98%, 99% or more sequence identity to at least one nucleotide sequence selected from the group consisting of SEQ ID NOS: 7, 8, 10, 11, 13, 14, 16, 17, 19, 20, 22, 23, 27, 28, 30, and 31.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, a polynucleotide that encodes a polypeptide with a given percent sequence identity to the polypeptide of SEQ ID NO: 9, 12, 15, 18, 21, 24, 29, and/or 32 is disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 60%, 65%, 70%, 75%, 80%, 85%, 86%, 87%, 88%, 89%, 90%, 91%, 92%, 93%, 94%, 95%, 96%, 97%, 98%, 99% or more sequence identity. In certain embodiments of the invention, variants of a particular polypeptide of the invention will have at least about 70%, 80%, 85%, 90%, 91%, 92%, 93%, 94%, 95%, 96%, 97%, 98%, 99% or more sequence identity to the amino acid sequence at least one amino acid sequence selected from the group consisting of the amino acid sequences set forth SEQ ID NO: 9, 12, 15, 18, 21, 24, 29, and 32.

“Variant” protein is intended to mean a protein derived from the a particular protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Such variants may result from, for example, genetic polymorphism or from human manipulation. Biologically active variants of a bs5 or bs5-L protein of the present invention will have at least about 70%, 80%, 85%, 86%, 87%, 88%, 89%, 90%, 91%, 92%, 93%, 94%, 95%, 96%, 97%, 98%, 99% or more sequence identity to the amino acid sequence for the native protein (e.g. the amino acid sequence set forth in SEQ ID NO: 9, 12, 15, 18, 21, 24, 29, or 32) as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. Methods for mutagenesis and polynucleotide alterations are well known in the art. See, for example, Kunkel (1985) PNAS 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

As discussed, the Bs5 genes that can be used in the methods of the present invention encompass orthologs of known Bs5 genes that occur in any plant species. Genes found in different species are considered orthologs when their nucleotide sequences and/or their encoded protein sequences share at least 60%, 70%, 75%, 80%, 85%, 86%, 87%, 88%, 89%, 90%, 91%, 92%, 93%, 94%, 95%, 96%, 97%, 98%, 99%, or greater sequence identity. Functions of orthologs are often highly conserved among species. Thus the orthologs of the present invention can have coding sequences comprising at least 70%, 80%, 85%, 86%, 87%, 88%, 89%, 90%, 91%, 92%, 93%, 94%, 95%, 96%, 97%, 98%, 99%, or greater nucleotide sequence identity to the nucleotide sequence of SIBs5 set forth in SEQ ID NO: 1, and/or encode proteins comprising at least 70, 80%, 85%, 86%, 87%, 88%, 89%, 90%, 91%, 92%, 93%, 94%, 95%, 96%, 97%, 98%, 99%, or greater amino acid sequence identity to the amino acid sequence set forth in SEQ ID NO: 3.

It is recognized that the mutated alleles and coding sequences of the present invention encompass polynucleotide molecules comprising a nucleotide sequence that is sufficiently identical to the nucleotide sequence of any one or more of SEQ ID NOS: 7, 8, 10, 11, 13, 14, 16, 17, 19, 20, 22, 23, 27, 29, 30, and 31. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 45%, 55%, or 65% identity, preferably 75% identity, more preferably 85%, 86%, 87%, 88%, 89%, 90%, 91%, 92%, 93%, 94%, 95%, 96%, 97%, 98% or 99% identity are defined herein as sufficiently identical.

To determine the percent identity of two amino acid sequences or of two nucleic acids, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are the same length. The percent identity between two sequences can be determined using techniques similar to those described below, with or without allowing gaps. In calculating percent identity, typically exact matches are counted.

The determination of percent identity between two sequences can be accomplished using a mathematical algorithm. A preferred, nonlimiting example of a mathematical algorithm utilized for the comparison of two sequences is the algorithm of Karlin and Altschul (1990) PNAS 87:2264, modified as in Karlin and Altschul (1993) PNAS 90:5873-5877. Such an algorithm is incorporated into the NBLAST and)(BLAST programs of Altschul et al. (1990) J Mol. Biol. 215:403. BLAST nucleotide searches can be performed with the NBLAST program, score=100, wordlength=12, to obtain nucleotide sequences homologous to the polynucleotide molecules of the invention. BLAST protein searches can be performed with the)(BLAST program, score=50, wordlength=3, to obtain amino acid sequences homologous to protein molecules of the invention. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g.,)(BLAST and NBLAST; available on the world-wide web at ncbi.nlm.nih.gov). Another preferred, non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used. Alignment may also be performed manually by inspection.

Unless otherwise stated, sequence identity/similarity values provided herein refer to the value obtained using the full-length sequences of the invention and using multiple alignment by mean of the algorithm Clustal W (Nucleic Acid Research, 22(22):4673-4680, 1994) using the program AlignX included in the software package Vector NTI Suite Version 7 (InforMax, Inc., Bethesda, Md., USA) using the default parameters; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by CLUSTALW (Version 1.83) using default parameters (available at the European Bioinformatics Institute website on the world-wide web at: ebi.ac.uk/Tools/clustalw/index.html).

The use of the term “polynucleotide” is not intended to limit the present invention to polynucleotides comprising DNA. Those of ordinary skill in the art will recognize that polynucleotides, can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The polynucleotide constructs comprising coding sequences of the present invention can be provided in expression cassettes for expression in the plant or other organism or non-human host cell of interest. The cassette will include 5′ and 3′ regulatory sequences operably linked to the coding sequence. “Operably linked” is intended to mean a functional linkage between two or more elements. For example, an operable linkage between a polynucleotide or gene of interest and a regulatory sequence (i.e., a promoter) is functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the coding sequence to be under the transcriptional regulation of the regulatory regions. The expression cassette may additionally contain selectable marker genes.

The expression cassette will include in the 5′-3′ direction of transcription, a transcriptional and translational initiation region (i.e., a promoter), a coding sequence of the invention, and a transcriptional and translational termination region (i.e., termination region) functional in plants or other organism or non-human host cell. The regulatory regions (i.e., promoters, transcriptional regulatory regions, and translational termination regions) and/or the coding sequence of the present invention may be native/analogous to the host cell or to each other. Alternatively, the regulatory regions and/or the coding sequence of the present invention may be heterologous to the host cell or to each other.

As used herein, “heterologous” in reference to a nucleic acid molecule or nucleotide sequence is a nucleic acid molecule or nucleotide sequence that originates from a foreign species, or, if from the same species, is modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

The present invention provides host cells comprising at least one of the nucleic acid molecules, expression cassettes, and vectors of the present invention. In preferred embodiments of the invention, a host cell is a plant cell. In other embodiments, a host cell is selected from the group consisting of a bacterium, a fungal cell, and an animal cell. In certain embodiments, a host cell is a non-human animal cell. However, in some other embodiments, the host cell is an in-vitro cultured human cell.

The termination region may be native with the transcriptional initiation region, may be native with the operably linked a coding sequence of interest, may be native with the plant host, or may be derived from another source (i.e., foreign or heterologous to the promoter, the coding sequence of interest, and/or the plant host), or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nuc. Acids Res. 17:7891-7903; and Joshi et al. (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) PNAS 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, tissue-preferred, or other promoters for expression in plants. Such constitutive promoters include, for example, the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Generally, it will be beneficial to express the gene from an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) PNAS 83: 2427-2430; Somsisch et al. (1988) Mol. Gen. Genet. 2:93-98; and Yang (1996) PNAS 93:14972-14977. See also, Chen et al. (1996) Plant J. 10:955-966; Zhang et al. (1994) PNAS 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant Path. 41:189-200).

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) PNAS 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) PNAS 86:5400-5404; Fuerst et al. (1989) PNAS 86:2549-2553; Deuschle et al. (1990) Science 248:480-483; Gossen (1993) Ph.D. Thesis, University of Heidelberg; Reines et al. (1993) PNAS 90:1917-1921; Labow et al. (1990) Mol. Cell. Biol. 10:3343-3356; Zambretti et al. (1992) PNAS 89:3952-3956; Baim et al. (1991) PNAS 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) PNAS 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not intended to be limiting. Any selectable marker gene can be used in the present invention.

Numerous plant transformation vectors and methods for transforming plants are available. See, for example, An et al. (1986) Plant Physiol., 81:301-305; Fry et al. (1987) Plant Cell Rep. 6:321-325; Block (1988) Theor. Appl. Genet. 76:767-774; Hinchee et al. (1990) Stadler Genet. Symp. 203212.203-212; Cousins, et al. (1991) Aust. J. Plant Physiol. 18:481-494; Chee and Slightom (1992) Gene. 118:255-260; Christou et al. (1992) Trends Biotechnol. 10:239-246; D'Halluin et al. (1992) Bio/Technol. 10:309-314; Dhir et al. (1992) Plant Physiol. 99:81-88; Casas et al. (1993) PNAS 90:11212-11216; Christou (1993) In Vitro Cell. Dev. Biol.-Plant; 29P:119-124; Davies et al. (1993) Plant Cell Rep. 12:180-183; Dongand Mchughen (1993) Plant Sci. 91:139-148; Franklin et al. (1993) Plant Cell Rep. 12(2):74-79, doi: 10.1007/BF00241938; Golovkin et al. (1993) Plant Sci. 90:41-52; Asano et al. (1994) Plant Cell Rep. 13; Ayeres and Park (1994) Crit. Rev. Plant Sci. 13:219-239; Barcelo et al. (1994) Plant J. 5:583-592; Becker et al. (1994) Plant J. 5:299-307; Borkowska et al. (1994) Acta Physiol. Plant 16:225-230; Christou (1994) Agro. Food Ind. Hi Tech. 5: 17-27; Eapen et al. (1994) Plant Cell Rep. 13:582-586; Hartman, et al. (1994) Bio-Technology 12: 919923; Ritala et al. (1994) Plant Mol. Biol. 24:317-325; and Wan and Lemaux (1994) Plant Physiol. 104:3748.

The methods of the invention can involve introducing a polynucleotide construct into a plant. By “introducing” is intended presenting to the plant the polynucleotide construct in such a manner that the construct gains access to the interior of a cell of the plant. The methods of the invention do not depend on a particular method for introducing a polynucleotide construct to a plant, only that the polynucleotide construct gains access to the interior of at least one cell of the plant. Methods for introducing polynucleotide constructs into plants are known in the art including, but not limited to, stable transformation methods, transient transformation methods, and virus-mediated methods.

By “stable transformation” is intended that the polynucleotide construct introduced into a plant integrates into the genome of the plant and is capable of being inherited by progeny thereof. By “transient transformation” is intended that a polynucleotide construct introduced into a plant does not integrate into the genome of the plant.

For the transformation of plants and plant cells, the nucleotide sequences of the invention are inserted using standard techniques into any vector known in the art that is suitable for expression of the nucleotide sequences in a plant or plant cell. The selection of the vector depends on the preferred transformation technique and the target plant species to be transformed.

Methodologies for constructing plant expression cassettes and introducing foreign nucleic acids into plants are generally known in the art and have been previously described. For example, foreign DNA can be introduced into plants, using tumor-inducing (Ti) plasmid vectors. Other methods utilized for foreign DNA delivery involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100: 247-250; Scheid et al., (1991) Mol. Gen. Genet., 228: 104-112; Guerche et al., (1987) Plant Science 52: 111-116; Neuhause et al., (1987) Theor. Appl Genet. 75: 30-36; Klein et al., (1987) Nature 327: 70-73; Howell et al., (1980) Science 208:1265; Horsch et al., (1985) Science 227: 1229-1231; DeBlock et al., (1989) Plant Physiology 91: 694-701; Methods for Plant Molecular Biology (Weissbach and Weissbach, eds.) Academic Press, Inc. (1988) and Methods in Plant Molecular Biology (Schuler and Zielinski, eds.) Academic Press, Inc. (1989). The method of transformation depends upon the plant cell to be transformed, stability of vectors used, expression level of gene products and other parameters.

Other suitable methods of introducing nucleotide sequences into plant cells and subsequent insertion into the plant genome include microinjection as Crossway et al. (1986) Biotechniques 4:320-334, electroporation as described by Riggs et al. (1986) PNAS 83:5602-5606, Agrobacterium-mediated transformation as described by Townsend et al., U.S. Pat. No. 5,563,055, Zhao et al., U.S. Pat. No. 5,981,840, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) PNAS 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) PNAS 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the invention may be introduced into plants by contacting plants with a virus or viral nucleic acids. Generally, such methods involve incorporating a polynucleotide construct of the invention within a viral DNA or RNA molecule. Further, it is recognized that promoters of the invention also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

If desired, the modified viruses or modified viral nucleic acids can be prepared in formulations. Such formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. Nos. 4,172,714, 4,144,050, 3,920,442, 5,180,587, 5,232,701, 5,208,030, GB 2,095,558, U.S. Pat. No. 3,299,566, Klingman, Weed Control as a Science, John Wiley and Sons, Inc., New York, 1961, Hance et al. Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, antifoaming agents, anti-freezing agents, for seed treatment formulation also optionally colorants and/or binders and/or gelling agents.

In specific embodiments, the polynucleotide constructs and expression cassettes of the invention can be provided to a plant using a variety of transient transformation methods known in the art. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) PNAS Sci. 91: 2176-2180 and Hush et al. (1994) J. Cell Science 107:775-784, all of which are herein incorporated by reference. Alternatively, the polynucleotide can be transiently transformed into the plant using techniques known in the art. Such techniques include viral vector system and Agrobacterium tumefaciens-mediated transient expression as described elsewhere herein.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a polynucleotide construct of the invention, for example, an expression cassette of the invention, stably incorporated into their genome.

The following example is offered by way of illustration and not by way of limitation.

### EXAMPLES

### Example 1: Inducing Mutations in SlBs5 Confers to Tomato Plants Resistance to Bacterial Pathogens

Because it was unknown whether the precise six-nucleotide deletion in the Cabs5 resistance allele of pepper is the sole mutation that confers resistance against bacterial spot. We set out to investigate whether introducing nucleotide insertions or deletions to the coding region of Bs5 would be sufficient to introduce resistance in tomato. Genome engineering in plants has recently been used as an alternative to plant breeding methods. Cas9 has emerged as a popular gene targeting tool due to its efficiency and sequence-specific DNA cleavage at target sites. Precise modifications in plant genomes has been achieved by exploiting error-prone DNA repair mechanisms after formation of Cas9-mediated double stranded breaks. In SlBs5/SlBs5-L, there is a protospacer-adjacent motif (PAM) in exon 2 within the proline-rich coding region, and there is a PAM in exon 3 following the six nucleotides that are deleted in the bs5 allele. We used these target sites for Cas9 mutagenesis to simultaneously target SlBs5/SlBs5-L and generate new alleles in the Bs5 gene. We aimed to test this allelic series of tomatoes for similar resistance phenotypes to that of bs5.

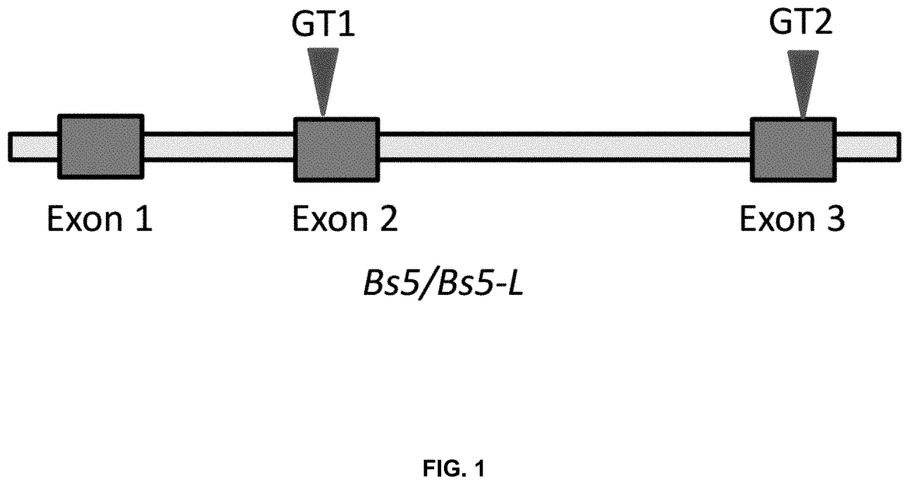

Lines of tomato with new Slbs5/Slbs5-L alleles were created using tomato varieties (M82 and FL8000) that are susceptible to X. euvesicatoria using a genome-editing approach. We created these lines by using an Agrobacterium transformation constructs containing the Cas9 gene driven by a Cauliflower Mosaic Virus (CaMV35S) constitutive promoter and single guide RNA targeting either the second or third exon (FIG. 1) of the tomato Bs5/Bs5-L genes (SlBs5|SlBs5-L). Agrobacterium-mediated tomato transformation of the construct was done in tissue culture of a susceptible tomato variety. Cotyledons from tomato seedlings were treated with a liquid culture of Agrobacterium containing the vector construct. Cotyledon were cultured on agar plates containing regeneration media to promote callus formation. Shoots were rooted and self-fertilized to obtain seeds of tomato plants with the Cas9-generated mutations. Three lines with unique mutation alleles were produced (FIGS. 2-3).

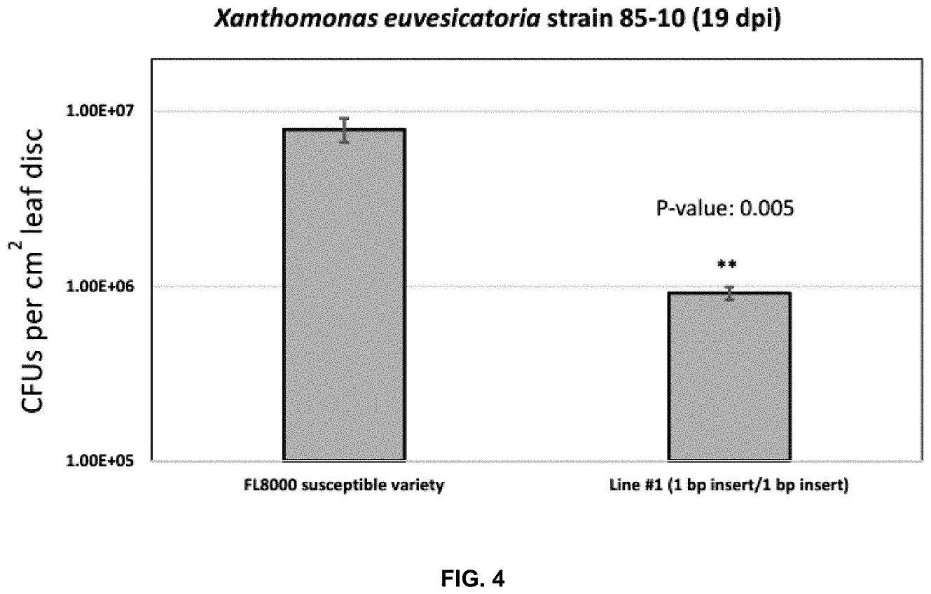

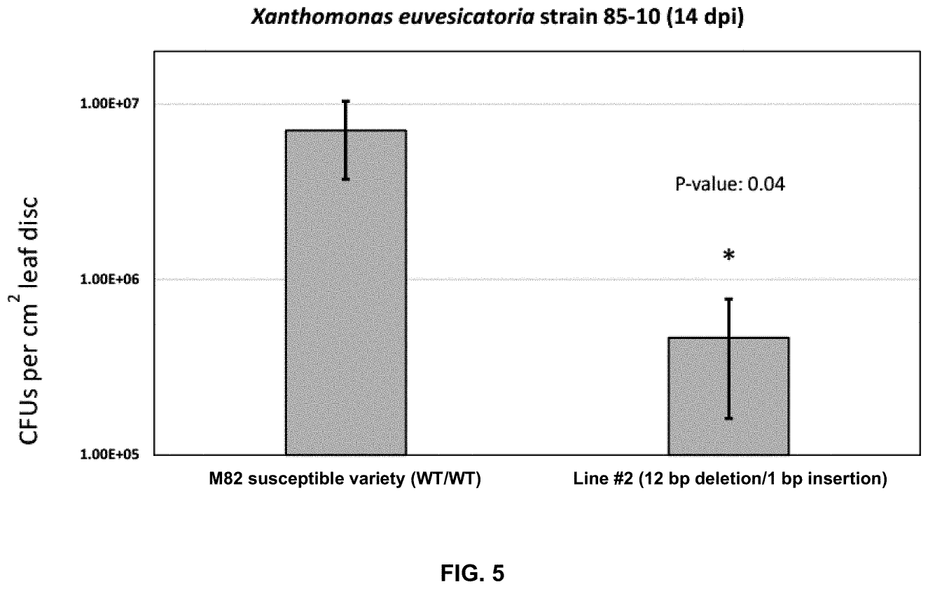

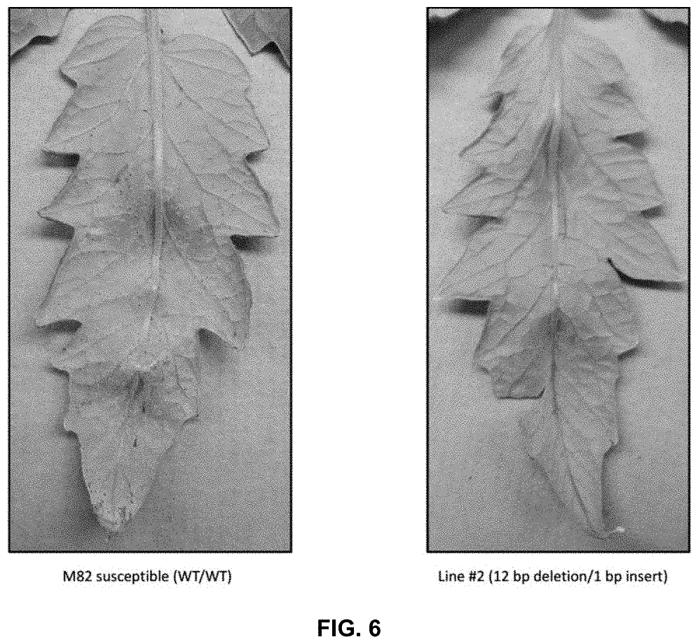

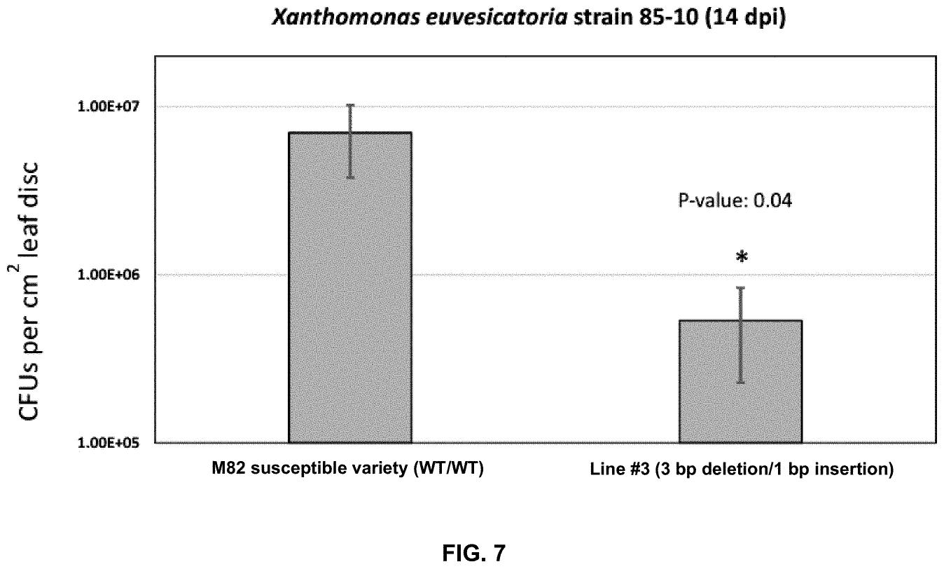

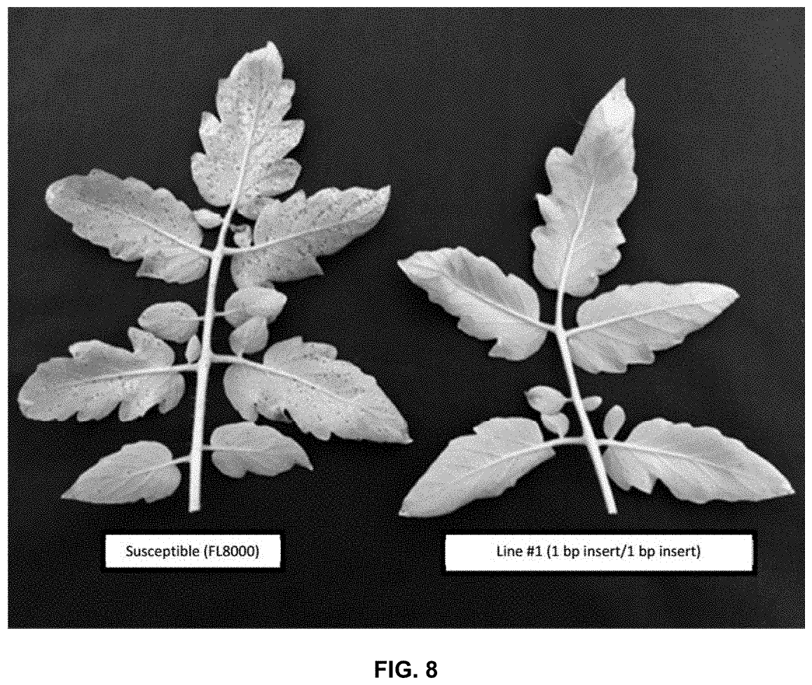

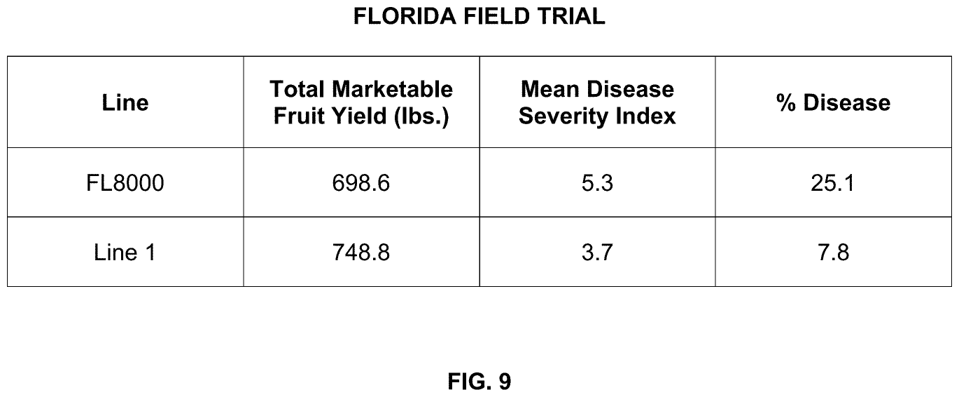

Pathogen challenges on these new lines were conducted to analyze resistance and compare the new lines with bs5 resistance pepper plants. Bacterial growth assays were done using X. euvesicatoria strain 85-10 (FIGS. 4-7), Xanthomonas perforans strain GEV345 (FIG. 6), and Pseudomonas syringae pv. tomato strain DC3000 (data not shown). One new line containing mutations in exon 2 (Line 1) showed resistance to not only X. euvesicatoria and X. perforans (FIG. 8), but also showed resistance to P. syringae pv. tomato. Line 1 also showed resistance in the field (FIG. 9), with no detrimental phenotypes. Two additional lines (Lines 2 and 3) with mutations in exon 3, also exhibited disease resistance against X. euvesicatoria.

### Example 2: Tomato Line 1 Displays Enhanced Resistance to Multiple Xanthomonas Species

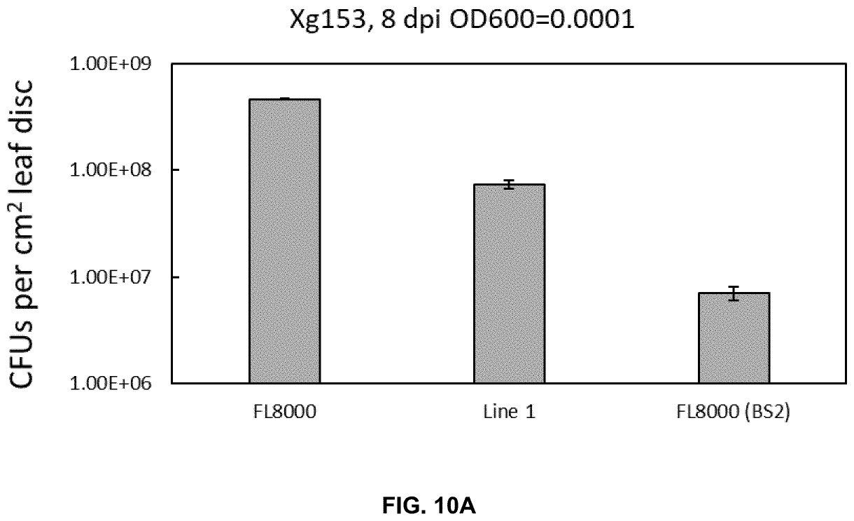

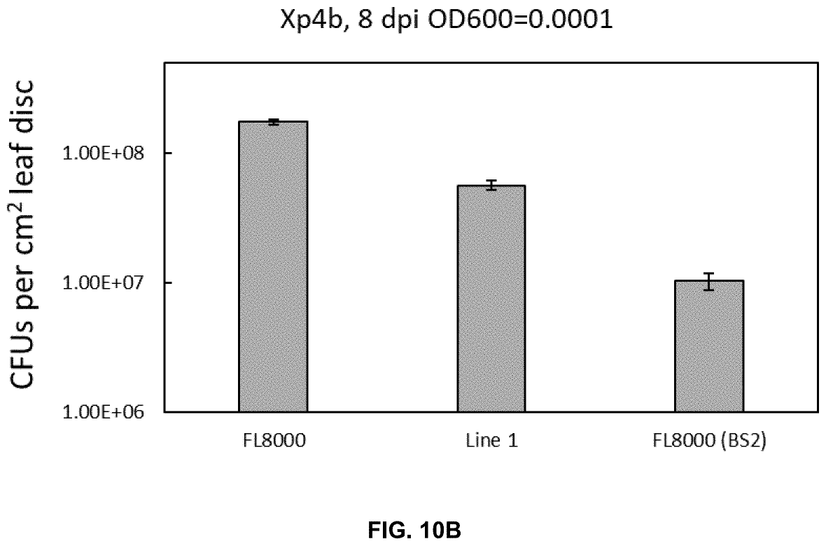

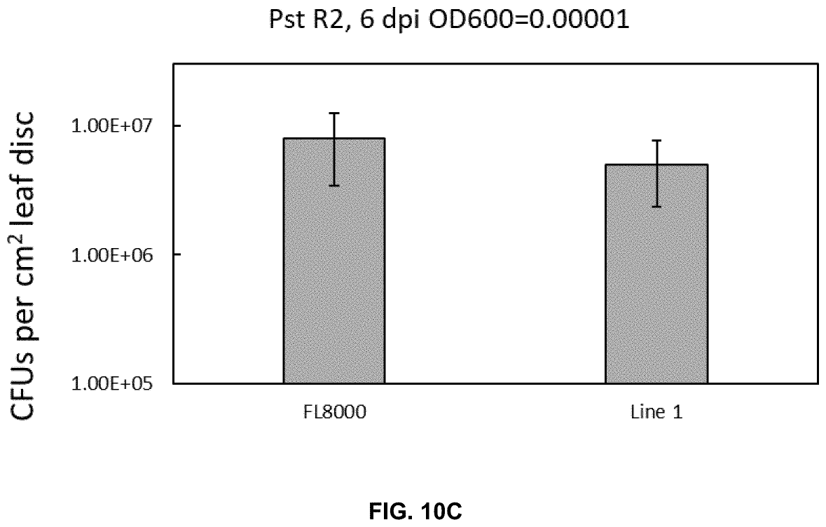

Additional pathogen challenges were conducted using tomato Line 1 and enhanced resistance to multiple Xanthomonas species was observed. Line 1 showed enhanced resistance to X. gardneri (Xg153) (FIG. 10A) and X. perforans (Xp4b) (FIG. 10B). A tomato line with the resistance gene BS2 (FL8000 BS2) was also included for comparison in FIGS. 10A and 10B. While Line 1 displayed resistance to Pseudomonas syringae pv. tomato strain DC3000 in a prior assay (Example 1), Line 1 was not significantly different from the susceptible control when inoculated with P. syringae (Pst R2) (FIG. 10C).

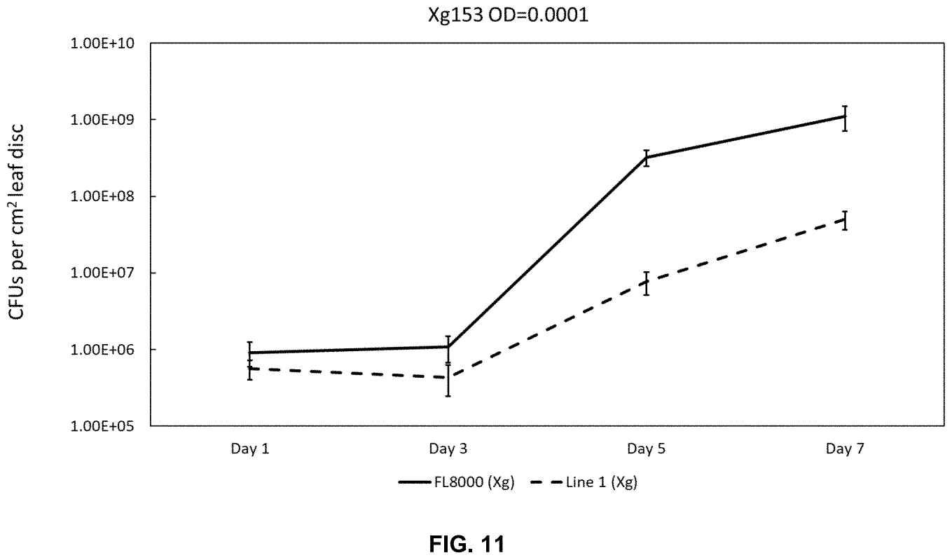

A bacterial growth time course study was conducted with Line 1 and a susceptible control line (FL8000) and the results are shown in FIG. 11. The susceptible wild-type tomato displayed a higher rate of bacterial growth over one week when compared to Line 1 (dashed line), resulting in about a 10-fold reduction in bacterial populations in Line 1 at Day 7.

### Example 3: Tomato Line 4 Displays Enhanced Resistance to Multiple Xanthomonas Species

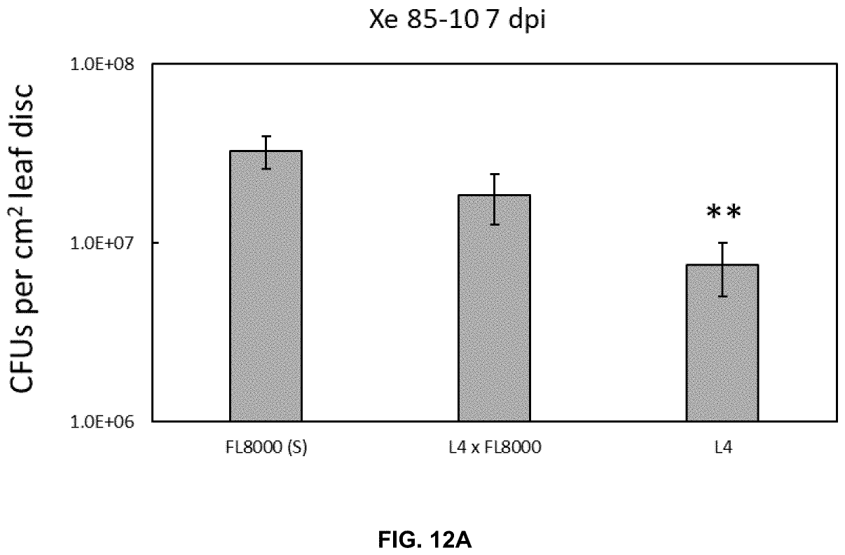

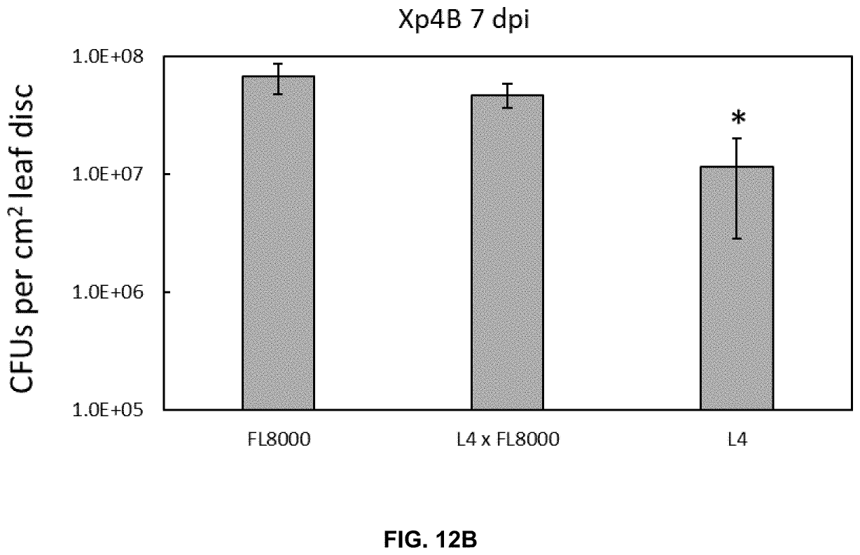

An additional tomato line (Line 4) with new Slbs5/Slbs5-L alleles was created from susceptible tomato variety FL8000 using the genome-editing approach described in Example 1 above. The mutations in Slbs5 and Slbs5-L alleles of Line 4 are both in exon 3 (FIGS. 2-3). Line 4 showed enhanced resistance to both X. euvesicatoria strain 85-10 or Xanthomonas X. perforans (Xp4b) when the tomato plants were homozygous for the new mutant alleles (FIGS. 12A-12B).

Pathogen challenges on these new lines were conducted to analyze resistance and compare the new lines with bs5 resistance pepper plants.

The article “a” and “an” are used herein to refer to one or more than one (i.e., to at least one) of the grammatical object of the article. By way of example, “an element” means one or more element.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A plant or seed with enhanced resistance to at least one plant pathogen, wherein the plant or seed comprises at least one mutated allele of a Bs5 and/or a Bs5-like (Bs5-L) gene, wherein the mutated allele comprises a mutation in the portion of the Bs5 or Bs5-L gene corresponding to exon 2 or exon 3 of the tomato Bs5 gene (SEQ ID NO: 1), wherein the at least one mutated allele is capable of conferring to the plant or seed enhanced resistance to at least one plant pathogen, relative to the resistance of a control plant or seed lacking the at least one mutated allele.

2. The plant or seed of claim 1, wherein the plant pathogen is selected from the group consisting of bacterial pathogens, oomycete pathogens, and fungal pathogens.

3. The plant or seed of claim 1, wherein the plant pathogen is selected from the group consisting of Xanthomonas spp., Pseudomonas spp., and Ralstonia spp.

4. The plant or seed claim 1, wherein the plant or seed comprises enhanced resistance to at least two, three, or four plant pathogens, relative to the resistance of a control plant or seed lacking the at least one mutated allele.

5. The plant or seed of claim 1, wherein the plant or seed is a solanaceous plant or seed.

6. The plant or seed of claim 1, wherein the plant or seed is a tomato plant or seed and the at least one mutated allele comprises a nucleotide sequence selected from the group consisting of:
(a) the nucleotide sequence set forth in SEQ ID NO: 7, 10, 13, 16, 19, 22, 27 or 30;
(b) a nucleotide sequence encoding a protein comprising the amino acid set forth in SEQ ID NO: 9, 12, 15, 18, 21, 24, 29, or 32;
(c) a nucleotide sequence having at least 90 or 95% nucleotide sequence identity to at least one nucleotide sequence of (a), wherein the nucleotide sequence is capable of conferring to a plant enhanced resistance to at least one plant pathogen; and
(d) a nucleotide sequence encoding a protein comprising an amino acid having at least 90 or 95% amino acid sequence identity to at least one of the amino acid sequences set forth in (a), wherein the nucleotide sequence is capable of conferring to a plant enhanced resistance to at least one plant pathogen.

7. A method for producing a plant with enhanced resistance to at least one plant pathogen, the method comprising:
(a) inducing a mutation at a specific location within at least one of the Bs5 and Bs5-L genes in the genome of a plant cell, whereby at least one mutated allele of the Bs5 gene or the Bs5-L gene is produced; and
(b) regenerating a plant from the plant cell, wherein the plant comprises in its genome the at least one mutated allele.

8. The method of claim 7, further comprising selfing the regenerated plant and selecting a progeny plant that is homozygous for the at least one mutated allele.

9. The method of claim 8, wherein the regenerated plant or the progeny plant comprises enhanced resistance to at least one plant pathogen, relative to the resistance of a control plant lacking the at least one mutated allele.

10. The method of claim 7, wherein (a) comprises inducing at least one single-strand or double-strand break at a specific location within at least one of the Bs5 and Bs5-L genes, whereby at least one mutated allele is produced.

11. The method of claim 10, wherein the single-strand or double-strand break is induced by a clustered regularly interspaced short palindromic repeats/CRISPR-associated nuclease (CRISPR/Cas nuclease), a transcription activator-like effector nuclease (TALEN), a zinc-finger nuclease (ZFN), or a homing endonuclease.

12. The method of claim 7, wherein the at least one mutated allele comprises the insertion of at least one nucleotide, the deletion of at least one nucleotide, or the insertion of at least one nucleotide and deletion of at least one nucleotide, relative to the nucleotide sequence of the Bs5 or Bs5-L gene.

13. The method of claim 7, wherein the specific location is a location with the Bs5 or Bs5-L gene corresponding to exon 2 or exon 3 of the tomato Bs5 gene (SEQ ID NO: 1).

14. The method of claim 7, wherein the plant is a solanaceous plant.

15. A plant produced or producible by the method of claim 7.

16. A mutated allele produced or producible by the method of claim 7; an isolated nucleic acid molecule comprising the mutated allele or comprising a nucleotide sequence encoding the protein encoded by the mutated allele; a host cell, plant, or seed comprising the mutated allele or isolated nucleic acid molecule; or a protein encoded by the mutated allele or isolated nucleic acid molecule.

17. An isolated nucleic acid molecule comprising a nucleotide sequence selected from the group consisting of:
(a) the nucleotide sequence set forth in SEQ ID NO: 7, 8, 10, 11, 13, 14, 16, 17, 19, 20, 22, 23, 27, 28, 30, or 31;
(b) a nucleotide sequence encoding a protein comprising the amino acid set forth in SEQ ID NO: 9, 12, 15, 18, 21, 24, 29, or 32;
(c) a nucleotide sequence having at least 80% nucleotide sequence identity to at least one nucleotide sequence of (a), wherein the nucleotide sequence is capable of conferring to a plant enhanced resistance to at least one plant pathogen; and
(d) a nucleotide sequence encoding a protein comprising an amino acid having at least 80% amino acid sequence identity to at least one of the amino acid sequences set forth in (a), wherein the nucleotide sequence is capable of conferring to a plant enhanced resistance to at least one plant pathogen.

18. A host cell, plant, or seed comprising the nucleic acid molecule of claim 17.

19. A protein encoded by the nucleic acid molecule or claim 17.

20. A method of limiting a plant disease caused by at least one plant pathogen in agricultural crop production, the method comprising planting a plant or seed of claim 1 and growing the plant or seed under conditions favorable for the growth and development of the plant.


SEQUENCE LISTING (32 sequences)

SEQ ID NO: 1 - DNA, 1981 bp, Solanum lycopersicum
ggagagtttcttggaatatgctgggcttagtccttgtttgacttgctctataaaacctaacaaataaaccccatttctttgtgcataagcaaaaactcaaaactctcttcaacaagaaatttcaattatgagttactacaatcagcagcaaccccctgttggtgtaccaccaccacaaggtaaattgcttcaatttcattttttttttgatgatttatggtaaagttcatatttttatatattgttttgatgatttatggtgaagttgatgtttttatatgttgttttgatgatttgtggtaaagttgatgtttttatatgttattttgatgattagatctgatggggttttggggaattttgtaattaggttatccaccagaaggttactctaaagatgcatacccaccaccagggtatcctcagcaagggtatccaccacagggttatcctcaacaagggtatccacctccacagtatgcacctcagtatggtgctccacctcctcaacaacatcaacagcaatctagtagcactggattaatgcaaggatggtatgcatctttacactcgtgatgttttgttatgatttagttgtgttgttgaattgtatgatctgagattttttttggattgagttgactatggatgataaggtgtgtaatgatttgattttgtcaactgaattgggtgaggttagtatgatctcgggttttgttgactaagttgccgagagacttgttcatatttgtgtgtctttccttgtttgtctcgaaaagatcttttgatcatcaaagggggaaagtagaagcaaggggggctagacctctagaccttaattgctgcatagattcatcatgttgctattagttgattttctttgaatattttagcaacctgtggattcgatataagcaatctaggtttttttttggatgaatttcttgaccagttttagtcgagagaagatcaaaggaatttggagaagaaatgtccttttggggcagaagctcgagcaatggaatatccctcgtgtgttacacattaacacgagttggagatccaaccgattctaatgcgtgaatattttttatgtgccaccatagtccggtcctttccattactgttatcaacgcccttcattttgtctcttgttttcattgtatctactgattcatcaccagtagcatgcaatggaattttcaacttgctgtacaatatcatgtttctcattaaaattctgccctgaatacactgctcaaattgccggtttagacttaacttgctttgtctatagctattgttttccaactgctgctcctatagataaaataatgacctcttagccaaatcagtctagagctaagtttggcaaagtaatccacactgccgccttagttgttgtttctttgtttctacctccaatatcctatgttgatgctccttcccattgatcaaaccttcaagttgtcgcagtgccaatgaactagtcagacaatgattcttgattccgtgatgtttcagtagcccttttttcttttagcactttgtcatattatttcgaatttccagtgggactaataatggtttgtggtattgttttgcagtttggctgctctgtgctgttgctgtctcttggatgcatgcttttgagggtgtaaatgatctgtgccatgtgttgatggcaaaagtttattgaatcaattatatcatagtctagacttttttctttctgtgttttgtcctatacttactttgataaataatttgatctttgatgtgctcaagattccaaagtgtttgttttgccaaattataggtggttggttatgtacaatgtgacagattctatttttatttttttcaatgttttggacctcaaaatattatatgtgaatcaatgcaccttgctctggttacaaatttatgctcctgttgtattaaatgtgttagcgaacacaacgtttgggttaaagccccg

SEQ ID NO: 2 - DNA, 279 bp, Solanum lycopersicum
atgagttactacaatcagcagcaaccccctgttggtgtaccaccaccacaaggttatccaccagaaggttactctaaagatgcatacccaccaccagggtatcctcagcaagggtatccaccacagggttatcctcaacaagggtatccacctccacagtatgcacctcagtatggtgctccacctcctcaacaacatcaacagcaatctagtagcactggattaatgcaaggatgcttggctgctctgtgctgttgctgtctcttggatgcatgcttt

SEQ ID NO: 3 - protein, 93 aa, Solanum lycopersicum
MSYYNQQQPPVGVPPPQGYPPEGYSKDAYPPPGYPQQGYPPQGYPQQGYPPPQYAPQYGAPPPQQHQQQSSSTGLMQGCLAALCCCCLLDACF

SEQ ID NO: 4 - DNA, 1625 bp, Solanum lycopersicum
cactgtgcagcccattatctttctttgtgtctaagcaaaagcaccaacagaaactctctctacagttagatccaacccaaattcttcattaattcaacaatgagttactacaatcaacaacaaccccctgttggtgtgccaccaccacaaggtaaatcacttcaattcctttttttctgattttattttggtaaagttgatgtttttatatgtttttttgatgactagatctgatggggttttgaatttttgtaggatatccgccagaaggttacccaaaagatgcatacccaccaccagggtacccacagcagggttaccctcaacaaggttacccacctcaagggtaccctccacagtatgcacctcagtatggtgctccacctcctcatcaacaacaacagcaatctggtactggtttcatggaaggatggtatgaaccttaaagactcaatttttatgttcatatgttatgattcagttacagtaatttgtatgattttgtttgagagagatctgtattgatattggttttttgtagagcggttgaggagattgtttatctgtcttgtctgttactatgtgtgtgtttcttgttgatttggtgtgtcttgtcaaaacatgatttctcgaattattattggtctgtttgatgtttactttgtgagcacttgtatttaataactatggttctggacgagttctggacgcgaactagaaggggtccttatcatttgtatgtctagttgaacattaacgcgagcaggagagacaattagaatgcctgaatgattctctatgtgcaaacatagtccaggccttgccacacctttgattttatctctgactgctgaaatcgcgcccctatcatacttcagactggaagggggtgcatggactagacataacttgtgttgctttgtctatagctataggttttccactgttgctgctgcaataagacaataatgacctcttatcaaagaaatctacactgtcacgttaattgtttcttcgttgtaatagactatgggagtatccttgttgatgctccattccactgtaagataagtagttaactgatctttcaaaacaatcacataattgtcatattaatcacacgtttaagttatcgcagtgccagtgaaccaattagacaatggacgtggtcttggctaccctaaattttcatagaatatagtggtggtctttcacatttcaacagctttgtgattttctgttttaggcgttccttctttcttgtagcctctcctcattttatttgagcttgctgtatttgttatttccggtgggggactaatcattggttgttatatttttgttttgcagtttggctgctctgtgctgttgctgtctcttggatgcatgcttttgatgctgtaaatgatctgtgccatgtgttggtggcaaaagtttattaaaccaattctatcatagtctagactttctctttttgtgtttgtctttggtgtcctgtactgtccttgataaataatttgatattaaatatgacgatgcaccttgttatggtgggagaattcaagtgtctatttgccaatttatacattattgtgcaatgcaagagattctatttttttaaaatc

SEQ ID NO: 5 - DNA, 273 bp, Solanum lycopersicum
atgagttactacaatcaacaacaaccccctgttggtgtgccaccaccacaaggatatccgccagaaggttacccaaaagatgcatacccaccaccagggtacccacagcagggttaccctcaacaaggttacccacctcaagggtaccctccacagtatgcacctcagtatggtgctccacctcctcatcaacaacaacagcaatctggtactggtttcatggaaggatgtttggctgctctgtgctgttgctgtctcttggatgcatgcttt

SEQ ID NO: 6 - protein, 91 aa, Solanum lycopersicum
MSYYNQQQPPVGVPPPQGYPPEGYPKDAYPPPGYPQQGYPQQGYPPQGYPPQYAPQYGAPPPHQQQQQSGTGFMEGCLAALCCCCLLDACF

SEQ ID NO: 7 - DNA, 1982 bp, Artificial Sequence
  Sequence resulting from in vivo targeted mutagenesis
ggagagtttcttggaatatgctgggcttagtccttgtttgacttgctctataaaacctaacaaataaaccccatttctttgtgcataagcaaaaactcaaaactctcttcaacaagaaatttcaattatgagttactacaatcagcagcaaccccctgttggtgtaccaccaccacaaggtaaattgcttcaatttcattttttttttgatgatttatggtaaagttcatatttttatatattgttttgatgatttatggtgaagttgatgtttttatatgttgttttgatgatttgtggtaaagttgatgtttttatatgttattttgatgattagatctgatggggttttggggaattttgtaattaggttatccaccagaaggttactctaaagatgcatacccaccaaccagggtatcctcagcaagggtatccaccacagggttatcctcaacaagggtatccacctccacagtatgcacctcagtatggtgctccacctcctcaacaacatcaacagcaatctagtagcactggattaatgcaaggatggtatgcatctttacactcgtgatgttttgttatgatttagttgtgttgttgaattgtatgatctgagattttttttggattgagttgactatggatgataaggtgtgtaatgatttgattttgtcaactgaattgggtgaggttagtatgatctcgggttttgttgactaagttgccgagagacttgttcatatttgtgtgtctttccttgtttgtctcgaaaagatcttttgatcatcaaagggggaaagtagaagcaaggggggctagacctctagaccttaattgctgcatagattcatcatgttgctattagttgattttctttgaatattttagcaacctgtggattcgatataagcaatctaggtttttttttggatgaatttcttgaccagttttagtcgagagaagatcaaaggaatttggagaagaaatgtccttttggggcagaagctcgagcaatggaatatccctcgtgtgttacacattaacacgagttggagatccaaccgattctaatgcgtgaatattttttatgtgccaccatagtccggtcctttccattactgttatcaacgcccttcattttgtctcttgttttcattgtatctactgattcatcaccagtagcatgcaatggaattttcaacttgctgtacaatatcatgtttctcattaaaattctgccctgaatacactgctcaaattgccggtttagacttaacttgctttgtctatagctattgttttccaactgctgctcctatagataaaataatgacctcttagccaaatcagtctagagctaagtttggcaaagtaatccacactgccgccttagttgttgtttctttgtttctacctccaatatcctatgttgatgctccttcccattgatcaaaccttcaagttgtcgcagtgccaatgaactagtcagacaatgattcttgattccgtgatgtttcagtagcccttttttcttttagcactttgtcatattatttcgaatttccagtgggactaataatggtttgtggtattgttttgcagtttggctgctctgtgctgttgctgtctcttggatgcatgcttttgagggtgtaaatgatctgtgccatgtgttgatggcaaaagtttattgaatcaattatatcatagtctagacttttttctttctgtgttttgtcctatacttactttgataaataatttgatctttgatgtgctcaagattccaaagtgtttgttttgccaaattataggtggttggttatgtacaatgtgacagattctatttttatttttttcaatgttttggacctcaaaatattatatgtgaatcaatgcaccttgctctggttacaaatttatgctcctgttgtattaaatgtgttagcgaacacaacgtttgggttaaagccccg

SEQ ID NO: 8 - DNA, 210 bp, Artificial Sequence
  Sequence resulting from in vivo targeted mutagenesis
atgagttactacaatcagcagcaaccccctgttggtgtaccaccaccacaaggttatccaccagaaggttactctaaagatgcatacccaccaaccagggtatcctcagcaagggtatccaccacagggttatcctcaacaagggtatccacctccacagtatgcacctcagtatggtgctccacctcctcaacaacatcaacagcaatc

SEQ ID NO: 9 - protein, 70 aa, Artificial Sequence
  Sequence resulting from in vivo targeted mutagenesis
MSYYNQQQPPVGVPPPQGYPPEGYSKDAYPPTRVSSARVSTTGLSSTRVSTSTVCTSVWCSTSSTTSTAI

SEQ ID NO: 10 - DNA, 1626 bp, Artificial Sequence
  Sequence resulting from in vivo targeted mutagenesis
cactgtgcagcccattatctttctttgtgtctaagcaaaagcaccaacagaaactctctctacagttagatccaacccaaattcttcattaattcaacaatgagttactacaatcaacaacaaccccctgttggtgtgccaccaccacaaggtaaatcacttcaattcctttttttctgattttattttggtaaagttgatgtttttatatgtttttttgatgactagatctgatggggttttgaatttttgtaggatatccgccagaaggttacccaaaagatgcatacccacctaccagggtacccacagcagggttaccctcaacaaggttacccacctcaagggtaccctccacagtatgcacctcagtatggtgctccacctcctcatcaacaacaacagcaatctggtactggtttcatggaaggatggtatgaaccttaaagactcaatttttatgttcatatgttatgattcagttacagtaatttgtatgattttgtttgagagagatctgtattgatattggttttttgtagagcggttgaggagattgtttatctgtcttgtctgttactatgtgtgtgtttcttgttgatttggtgtgtcttgtcaaaacatgatttctcgaattattattggtctgtttgatgtttactttgtgagcacttgtatttaataactatggttctggacgagttctggacgcgaactagaaggggtccttatcatttgtatgtctagttgaacattaacgcgagcaggagagacaattagaatgcctgaatgattctctatgtgcaaacatagtccaggccttgccacacctttgattttatctctgactgctgaaatcgcgcccctatcatacttcagactggaagggggtgcatggactagacataacttgtgttgctttgtctatagctataggttttccactgttgctgctgcaataagacaataatgacctcttatcaaagaaatctacactgtcacgttaattgtttcttcgttgtaatagactatgggagtatccttgttgatgctccattccactgtaagataagtagttaactgatctttcaaaacaatcacataattgtcatattaatcacacgtttaagttatcgcagtgccagtgaaccaattagacaatggacgtggtcttggctaccctaaattttcatagaatatagtggtggtctttcacatttcaacagctttgtgattttctgttttaggcgttccttctttcttgtagcctctcctcattttatttgagcttgctgtatttgttatttccggtgggggactaatcattggttgttatatttttgttttgcagtttggctgctctgtgctgttgctgtctcttggatgcatgcttttgatgctgtaaatgatctgtgccatgtgttggtggcaaaagtttattaaaccaattctatcatagtctagactttctctttttgtgtttgtctttggtgtcctgtactgtccttgataaataatttgatattaaatatgacgatgcaccttgttatggtgggagaattcaagtgtctatttgccaatttatacattattgtgcaatgcaagagattctatttttttaaaatc

SEQ ID NO: 11 - DNA, 282 bp, Artificial Sequence
  Sequence resulting from in vivo targeted mutagenesis
atgagttactacaatcaacaacaaccccctgttggtgtgccaccaccacaaggatatccgccagaaggttacccaaaagatgcatacccacctaccagggtacccacagcagggttaccctcaacaaggttacccacctcaagggtaccctccacagtatgcacctcagtatggtgctccacctcctcatcaacaacaacagcaatctggtactggtttcatggaaggatgtttggctgctctgtgctgttgctgtctcttggatgcatgcttttgatgctg

SEQ ID NO: 12 - protein, 94 aa, Artificial Sequence
  Sequence resulting from in vivo targeted mutagenesis
MSYYNQQQPPVGVPPPQGYPPEGYPKDAYPPTRVPTAGLPSTRLPTSRVPSTVCTSVWCSTSSSTTTAIWYWFHGRMFGCSVLLLSLGCMLLML

SEQ ID NO: 13 - DNA, 1969 bp, Artificial Sequence
  Sequence resulting from in vivo targeted mutagenesis
ggagagtttcttggaatatgctgggcttagtccttgtttgacttgctctataaaacctaacaaataaaccccatttctttgtgcataagcaaaaactcaaaactctcttcaacaagaaatttcaattatgagttactacaatcagcagcaaccccctgttggtgtaccaccaccacaaggtaaattgcttcaatttcattttttttttgatgatttatggtaaagttcatatttttatatattgttttgatgatttatggtgaagttgatgtttttatatgttgttttgatgatttgtggtaaagttgatgtttttatatgttattttgatgattagatctgatggggttttggggaattttgtaattaggttatccaccagaaggttactctaaagatgcatacccaccaccagggtatcctcagcaagggtatccaccacagggttatcctcaacaagggtatccacctccacagtatgcacctcagtatggtgctccacctcctcaacaacatcaacagcaatctagtagcactggattaatgcaaggatggtatgcatctttacactcgtgatgttttgttatgatttagttgtgttgttgaattgtatgatctgagattttttttggattgagttgactatggatgataaggtgtgtaatgatttgattttgtcaactgaattgggtgaggttagtatgatctcgggttttgttgactaagttgccgagagacttgttcatatttgtgtgtctttccttgtttgtctcgaaaagatcttttgatcatcaaagggggaaagtagaagcaaggggggctagacctctagaccttaattgctgcatagattcatcatgttgctattagttgattttctttgaatattttagcaacctgtggattcgatataagcaatctaggtttttttttggatgaatttcttgaccagttttagtcgagagaagatcaaaggaatttggagaagaaatgtccttttggggcagaagctcgagcaatggaatatccctcgtgtgttacacattaacacgagttggagatccaaccgattctaatgcgtgaatattttttatgtgccaccatagtccggtcctttccattactgttatcaacgcccttcattttgtctcttgttttcattgtatctactgattcatcaccagtagcatgcaatggaattttcaacttgctgtacaatatcatgtttctcattaaaattctgccctgaatacactgctcaaattgccggtttagacttaacttgctttgtctatagctattgttttccaactgctgctcctatagataaaataatgacctcttagccaaatcagtctagagctaagtttggcaaagtaatccacactgccgccttagttgttgtttctttgtttctacctccaatatcctatgttgatgctccttcccattgatcaaaccttcaagttgtcgcagtgccaatgaactagtcagacaatgattcttgattccgtgatgtttcagtagcccttttttcttttagcactttgtcatattatttcgaatttccagtgggactaataatggtttgtggtattgttttgcagtttggctgctctgttcttggatgcatgcttttgagggtgtaaatgatctgtgccatgtgttgatggcaaaagtttattgaatcaattatatcatagtctagacttttttctttctgtgttttgtcctatacttactttgataaataatttgatctttgatgtgctcaagattccaaagtgtttgttttgccaaattataggtggttggttatgtacaatgtgacagattctatttttatttttttcaatgttttggacctcaaaatattatatgtgaatcaatgcaccttgctctggttacaaatttatgctcctgttgtattaaatgtgttagcgaacacaacgtttgggttaaagccccg

SEQ ID NO: 14 - DNA, 267 bp, Artificial Sequence
  Sequence resulting from in vivo targeted mutagenesis
atgagttactacaatcagcagcaaccccctgttggtgtaccaccaccacaaggttatccaccagaaggttactctaaagatgcatacccaccaccagggtatcctcagcaagggtatccaccacagggttatcctcaacaagggtatccacctccacagtatgcacctcagtatggtgctccacctcctcaacaacatcaacagcaatctagtagcactggattaatgcaaggatgcttggctgctctgttcttggatgcatgcttt

SEQ ID NO: 15 - protein, 89 aa, Artificial Sequence
  Sequence resulting from in vivo targeted mutagenesis
MSYYNQQQPPVGVPPPQGYPPEGYSKDAYPPPGYPQQGYPPQGYPQQGYPPPQYAPQYGAPPPQQHQQQSSSTGLMQGCLAALFLDACF

SEQ ID NO: 16 - DNA, 1626 bp, Artificial Sequence
  Sequence resulting from in vivo targeted mutagenesis
cactgtgcagcccattatctttctttgtgtctaagcaaaagcaccaacagaaactctctctacagttagatccaacccaaattcttcattaattcaacaatgagttactacaatcaacaacaaccccctgttggtgtgccaccaccacaaggtaaatcacttcaattcctttttttctgattttattttggtaaagttgatgtttttatatgtttttttgatgactagatctgatggggttttgaatttttgtaggatatccgccagaaggttacccaaaagatgcatacccaccaccagggtacccacagcagggttaccctcaacaaggttacccacctcaagggtaccctccacagtatgcacctcagtatggtgctccacctcctcatcaacaacaacagcaatctggtactggtttcatggaaggatggtatgaaccttaaagactcaatttttatgttcatatgttatgattcagttacagtaatttgtatgattttgtttgagagagatctgtattgatattggttttttgtagagcggttgaggagattgtttatctgtcttgtctgttactatgtgtgtgtttcttgttgatttggtgtgtcttgtcaaaacatgatttctcgaattattattggtctgtttgatgtttactttgtgagcacttgtatttaataactatggttctggacgagttctggacgcgaactagaaggggtccttatcatttgtatgtctagttgaacattaacgcgagcaggagagacaattagaatgcctgaatgattctctatgtgcaaacatagtccaggccttgccacacctttgattttatctctgactgctgaaatcgcgcccctatcatacttcagactggaagggggtgcatggactagacataacttgtgttgctttgtctatagctataggttttccactgttgctgctgcaataagacaataatgacctcttatcaaagaaatctacactgtcacgttaattgtttcttcgttgtaatagactatgggagtatccttgttgatgctccattccactgtaagataagtagttaactgatctttcaaaacaatcacataattgtcatattaatcacacgtttaagttatcgcagtgccagtgaaccaattagacaatggacgtggtcttggctaccctaaattttcatagaatatagtggtggtctttcacatttcaacagctttgtgattttctgttttaggcgttccttctttcttgtagcctctcctcattttatttgagcttgctgtatttgttatttccggtgggggactaatcattggttgttatatttttgttttgcagtttggctgctctgtgctgttgctgtcctcttggatgcatgcttttgatgctgtaaatgatctgtgccatgtgttggtggcaaaagtttattaaaccaattctatcatagtctagactttctctttttgtgtttgtctttggtgtcctgtactgtccttgataaataatttgatattaaatatgacgatgcaccttgttatggtgggagaattcaagtgtctatttgccaatttatacattattgtgcaatgcaagagattctatttttttaaaatc

SEQ ID NO: 17 - DNA, 282 bp, Artificial Sequence
  Sequence resulting from in vivo targeted mutagenesis
atgagttactacaatcaacaacaaccccctgttggtgtgccaccaccacaaggatatccgccagaaggttacccaaaagatgcatacccaccaccagggtacccacagcagggttaccctcaacaaggttacccacctcaagggtaccctccacagtatgcacctcagtatggtgctccacctcctcatcaacaacaacagcaatctggtactggtttcatggaaggatgtttggctgctctgtgctgttgctgtcctcttggatgcatgcttttgatgctg

SEQ ID NO: 18 - protein, 94 aa, Artificial Sequence
  Sequence resulting from in vivo targeted mutagenesis
MSYYNQQQPPVGVPPPQGYPPEGYPKDAYPPPGYPQQGYPQQGYPPQGYPPQYAPQYGAPPPHQQQQQSGTGFMEGCLAALCCCCLLGCMLLML

SEQ ID NO: 19 - DNA, 1978 bp, Artificial Sequence
  Sequence resulting from in vivo targeted mutagenesis
ggagagtttcttggaatatgctgggcttagtccttgtttgacttgctctataaaacctaacaaataaaccccatttctttgtgcataagcaaaaactcaaaactctcttcaacaagaaatttcaattatgagttactacaatcagcagcaaccccctgttggtgtaccaccaccacaaggtaaattgcttcaatttcattttttttttgatgatttatggtaaagttcatatttttatatattgttttgatgatttatggtgaagttgatgtttttatatgttgttttgatgatttgtggtaaagttgatgtttttatatgttattttgatgattagatctgatggggttttggggaattttgtaattaggttatccaccagaaggttactctaaagatgcatacccaccaccagggtatcctcagcaagggtatccaccacagggttatcctcaacaagggtatccacctccacagtatgcacctcagtatggtgctccacctcctcaacaacatcaacagcaatctagtagcactggattaatgcaaggatggtatgcatctttacactcgtgatgttttgttatgatttagttgtgttgttgaattgtatgatctgagattttttttggattgagttgactatggatgataaggtgtgtaatgatttgattttgtcaactgaattgggtgaggttagtatgatctcgggttttgttgactaagttgccgagagacttgttcatatttgtgtgtctttccttgtttgtctcgaaaagatcttttgatcatcaaagggggaaagtagaagcaaggggggctagacctctagaccttaattgctgcatagattcatcatgttgctattagttgattttctttgaatattttagcaacctgtggattcgatataagcaatctaggtttttttttggatgaatttcttgaccagttttagtcgagagaagatcaaaggaatttggagaagaaatgtccttttggggcagaagctcgagcaatggaatatccctcgtgtgttacacattaacacgagttggagatccaaccgattctaatgcgtgaatattttttatgtgccaccatagtccggtcctttccattactgttatcaacgcccttcattttgtctcttgttttcattgtatctactgattcatcaccagtagcatgcaatggaattttcaacttgctgtacaatatcatgtttctcattaaaattctgccctgaatacactgctcaaattgccggtttagacttaacttgctttgtctatagctattgttttccaactgctgctcctatagataaaataatgacctcttagccaaatcagtctagagctaagtttggcaaagtaatccacactgccgccttagttgttgtttctttgtttctacctccaatatcctatgttgatgctccttcccattgatcaaaccttcaagttgtcgcagtgccaatgaactagtcagacaatgattcttgattccgtgatgtttcagtagcccttttttcttttagcactttgtcatattatttcgaatttccagtgggactaataatggtttgtggtattgttttgcagtttggctgctctgtgctgttgcttcttggatgcatgcttttgagggtgtaaatgatctgtgccatgtgttgatggcaaaagtttattgaatcaattatatcatagtctagacttttttctttctgtgttttgtcctatacttactttgataaataatttgatctttgatgtgctcaagattccaaagtgtttgttttgccaaattataggtggttggttatgtacaatgtgacagattctatttttatttttttcaatgttttggacctcaaaatattatatgtgaatcaatgcaccttgctctggttacaaatttatgctcctgttgtattaaatgtgttagcgaacacaacgtttgggttaaagccccg

SEQ ID NO: 20 - DNA, 276 bp, Artificial Sequence
  Sequence resulting from in vivo targeted mutagenesis
atgagttactacaatcagcagcaaccccctgttggtgtaccaccaccacaaggttatccaccagaaggttactctaaagatgcatacccaccaccagggtatcctcagcaagggtatccaccacagggttatcctcaacaagggtatccacctccacagtatgcacctcagtatggtgctccacctcctcaacaacatcaacagcaatctagtagcactggattaatgcaaggatgcttggctgctctgtgctgttgcttcttggatgcatgcttt

SEQ ID NO: 21 - protein, 92 aa, Artificial Sequence
  Sequence resulting from in vivo targeted mutagenesis
MSYYNQQQPPVGVPPPQGYPPEGYSKDAYPPPGYPQQGYPPQGYPQQGYPPPQYAPQYGAPPPQQHQQQSSSTGLMQGCLAALCCCFLDACF

SEQ ID NO: 22 - DNA, 1626 bp, Artificial Sequence
  Sequence resulting from in vivo targeted mutagenesis
cactgtgcagcccattatctttctttgtgtctaagcaaaagcaccaacagaaactctctctacagttagatccaacccaaattcttcattaattcaacaatgagttactacaatcaacaacaaccccctgttggtgtgccaccaccacaaggtaaatcacttcaattcctttttttctgattttattttggtaaagttgatgtttttatatgtttttttgatgactagatctgatggggttttgaatttttgtaggatatccgccagaaggttacccaaaagatgcatacccaccaccagggtacccacagcagggttaccctcaacaaggttacccacctcaagggtaccctccacagtatgcacctcagtatggtgctccacctcctcatcaacaacaacagcaatctggtactggtttcatggaaggatggtatgaaccttaaagactcaatttttatgttcatatgttatgattcagttacagtaatttgtatgattttgtttgagagagatctgtattgatattggttttttgtagagcggttgaggagattgtttatctgtcttgtctgttactatgtgtgtgtttcttgttgatttggtgtgtcttgtcaaaacatgatttctcgaattattattggtctgtttgatgtttactttgtgagcacttgtatttaataactatggttctggacgagttctggacgcgaactagaaggggtccttatcatttgtatgtctagttgaacattaacgcgagcaggagagacaattagaatgcctgaatgattctctatgtgcaaacatagtccaggccttgccacacctttgattttatctctgactgctgaaatcgcgcccctatcatacttcagactggaagggggtgcatggactagacataacttgtgttgctttgtctatagctataggttttccactgttgctgctgcaataagacaataatgacctcttatcaaagaaatctacactgtcacgttaattgtttcttcgttgtaatagactatgggagtatccttgttgatgctccattccactgtaagataagtagttaactgatctttcaaaacaatcacataattgtcatattaatcacacgtttaagttatcgcagtgccagtgaaccaattagacaatggacgtggtcttggctaccctaaattttcatagaatatagtggtggtctttcacatttcaacagctttgtgattttctgttttaggcgttccttctttcttgtagcctctcctcattttatttgagcttgctgtatttgttatttccggtgggggactaatcattggttgttatatttttgttttgcagtttggctgctctgtgctgttgctgctttcttggatgcatgcttttgatgctgtaaatgatctgtgccatgtgttggtggcaaaagtttattaaaccaattctatcatagtctagactttctctttttgtgtttgtctttggtgtcctgtactgtccttgataaataatttgatattaaatatgacgatgcaccttgttatggtgggagaattcaagtgtctatttgccaatttatacattattgtgcaatgcaagagattctatttttttaaaatc

SEQ ID NO: 23 - DNA, 282 bp, Artificial Sequence
  Sequence resulting from in vivo targeted mutagenesis
atgagttactacaatcaacaacaaccccctgttggtgtgccaccaccacaaggatatccgccagaaggttacccaaaagatgcatacccaccaccagggtacccacagcagggttaccctcaacaaggttacccacctcaagggtaccctccacagtatgcacctcagtatggtgctccacctcctcatcaacaacaacagcaatctggtactggtttcatggaaggatgtttggctgctctgtgctgttgctgctttcttggatgcatgcttttgatgctg

SEQ ID NO: 24 - protein, 94 aa, Artificial Sequence
  Sequence resulting from in vivo targeted mutagenesis
MSYYNQQQPPVGVPPPQGYPPEGYPKDAYPPPGYPQQGYPQQGYPPQGYPPQYAPQYGAPPPHQQQQQSGTGFMEGCLAALCCCCFLGCMLLML

SEQ ID NO: 25 - DNA, 23 bp, Solanum lycopersicum
aagatgcatacccaccaccaggg

SEQ ID NO: 26 - DNA, 23 bp, Solanum lycopersicum
tctgtgctgttgctgtctcttgg

SEQ ID NO: 27 - DNA, 1974 bp, Artificial Sequence
  Sequence resulting from in vivo targeted mutagenesis
ggagagtttcttggaatatgctgggcttagtccttgtttgacttgctctataaaacctaacaaataaaccccatttctttgtgcataagcaaaaactcaaaactctcttcaacaagaaatttcaattatgagttactacaatcagcagcaaccccctgttggtgtaccaccaccacaaggtaaattgcttcaatttcattttttttttgatgatttatggtaaagttcatatttttatatattgttttgatgatttatggtgaagttgatgtttttatatgttgttttgatgatttgtggtaaagttgatgtttttatatgttattttgatgattagatctgatggggttttggggaattttgtaattaggttatccaccagaaggttactctaaagatgcatacccaccaccagggtatcctcagcaagggtatccaccacagggttatcctcaacaagggtatccacctccacagtatgcacctcagtatggtgctccacctcctcaacaacatcaacagcaatctagtagcactggattaatgcaaggatggtatgcatctttacactcgtgatgttttgttatgatttagttgtgttgttgaattgtatgatctgagattttttttggattgagttgactatggatgataaggtgtgtaatgatttgattttgtcaactgaattgggtgaggttagtatgatctcgggttttgttgactaagttgccgagagacttgttcatatttgtgtgtctttccttgtttgtctcgaaaagatcttttgatcatcaaagggggaaagtagaagcaaggggggctagacctctagaccttaattgctgcatagattcatcatgttgctattagttgattttctttgaatattttagcaacctgtggattcgatataagcaatctaggtttttttttggatgaatttcttgaccagttttagtcgagagaagatcaaaggaatttggagaagaaatgtccttttggggcagaagctcgagcaatggaatatccctcgtgtgttacacattaacacgagttggagatccaaccgattctaatgcgtgaatattttttatgtgccaccatagtccggtcctttccattactgttatcaacgcccttcattttgtctcttgttttcattgtatctactgattcatcaccagtagcatgcaatggaattttcaacttgctgtacaatatcatgtttctcattaaaattctgccctgaatacactgctcaaattgccggtttagacttaacttgctttgtctatagctattgttttccaactgctgctcctatagataaaataatgacctcttagccaaatcagtctagagctaagtttggcaaagtaatccacactgccgccttagttgttgtttctttgtttctacctccaatatcctatgttgatgctccttcccattgatcaaaccttcaagttgtcgcagtgccaatgaactagtcagacaatgattcttgattccgtgatgtttcagtagcccttttttcttttagcactttgtcatattatttcgaatttccagtgggactaataatggtttgtggtattgttttgcagtttggctgctctgtgctgttcttggatgcatgcttttgagggtgtaaatgatctgtgccatgtgttgatggcaaaagtttattgaatcaattatatcatagtctagacttttttctttctgtgttttgtcctatacttactttgataaataatttgatctttgatgtgctcaagattccaaagtgtttgttttgccaaattataggtggttggttatgtacaatgtgacagattctatttttatttttttcaatgttttggacctcaaaatattatatgtgaatcaatgcaccttgctctggttacaaatttatgctcctgttgtattaaatgtgttagcgaacacaacgtttgggttaaagccccg

SEQ ID NO: 28 - DNA, 321 bp, Artificial Sequence
  Sequence resulting from in vivo targeted mutagenesis
atgagttactacaatcagcagcaaccccctgttggtgtaccaccaccacaaggttatccaccagaaggttactctaaagatgcatacccaccaccagggtatcctcagcaagggtatccaccacagggttatcctcaacaagggtatccacctccacagtatgcacctcagtatggtgctccacctcctcaacaacatcaacagcaatctagtagcactggattaatgcaaggatgcttggctgctctgtgctgttcttggatgcatgcttttgagggtgtaaatgatctgtgccatgtgttgatggcaaaagtttattga

SEQ ID NO: 29 - protein, 106 aa, Artificial Sequence
  Sequence resulting from in vivo targeted mutagenesis
MSYYNQQQPPVGVPPPQGYPPEGYSKDAYPPPGYPQQGYPPQGYPQQGYPPPQYAPQYGAPPPQQHQQQSSSTGLMQGCLAALCCSWMHAFEGVNDLCHVLMAKVY

SEQ ID NO: 30 - DNA, 1626 bp, Artificial Sequence
  Sequence resulting from in vivo targeted mutagenesis
cactgtgcagcccattatctttctttgtgtctaagcaaaagcaccaacagaaactctctctacagttagatccaacccaaattcttcattaattcaacaatgagttactacaatcaacaacaaccccctgttggtgtgccaccaccacaaggtaaatcacttcaattcctttttttctgattttattttggtaaagttgatgtttttatatgtttttttgatgactagatctgatggggttttgaatttttgtaggatatccgccagaaggttacccaaaagatgcatacccaccaccagggtacccacagcagggttaccctcaacaaggttacccacctcaagggtaccctccacagtatgcacctcagtatggtgctccacctcctcatcaacaacaacagcaatctggtactggtttcatggaaggatggtatgaaccttaaagactcaatttttatgttcatatgttatgattcagttacagtaatttgtatgattttgtttgagagagatctgtattgatattggttttttgtagagcggttgaggagattgtttatctgtcttgtctgttactatgtgtgtgtttcttgttgatttggtgtgtcttgtcaaaacatgatttctcgaattattattggtctgtttgatgtttactttgtgagcacttgtatttaataactatggttctggacgagttctggacgcgaactagaaggggtccttatcatttgtatgtctagttgaacattaacgcgagcaggagagacaattagaatgcctgaatgattctctatgtgcaaacatagtccaggccttgccacacctttgattttatctctgactgctgaaatcgcgcccctatcatacttcagactggaagggggtgcatggactagacataacttgtgttgctttgtctatagctataggttttccactgttgctgctgcaataagacaataatgacctcttatcaaagaaatctacactgtcacgttaattgtttcttcgttgtaatagactatgggagtatccttgttgatgctccattccactgtaagataagtagttaactgatctttcaaaacaatcacataattgtcatattaatcacacgtttaagttatcgcagtgccagtgaaccaattagacaatggacgtggtcttggctaccctaaattttcatagaatatagtggtggtctttcacatttcaacagctttgtgattttctgttttaggcgttccttctttcttgtagcctctcctcattttatttgagcttgctgtatttgttatttccggtgggggactaatcattggttgttatatttttgttttgcagtttggctgctctgtgctgttgctgtctcttgggatgcatgcttttgatgctgtaaatgatctgtgccatgtgttggtggcaaaagtttattaaaccaattctatcatagtctagactttctctttttgtgtttgtctttggtgtcctgtactgtccttgataaataatttgatattaaatatgacgatgcaccttgttatggtgggagaattcaagtgtctatttgccaatttatacattattgtgcaatgcaagagattctatttttttaaaatc

SEQ ID NO: 31 - DNA, 285 bp, Artificial Sequence
  Sequence resulting from in vivo targeted mutagenesis
atgagttactacaatcaacaacaaccccctgttggtgtgccaccaccacaaggatatccgccagaaggttacccaaaagatgcatacccaccaccagggtacccacagcagggttaccctcaacaaggttacccacctcaagggtaccctccacagtatgcacctcagtatggtgctccacctcctcatcaacaacaacagcaatctggtactggtttcatggaaggatgtttggctgctctgtgctgttgctgtctcttgggatgcatgcttttgatgctgtaa

SEQ ID NO: 32 - protein, 94 aa, Artificial Sequence; identical to SEQ ID NO: 18
  Sequence resulting from in vivo targeted mutagenesis